SEQUENCE LISTING (3 sequences)

SEQ ID NO: 1 - protein, 1210 aa, Homo sapiens [sequence omitted]

SEQ ID NO: 2 - protein, 1186 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 3 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

(19) United States
(12) Patent Application Publication
(10) Pub. No.: US 2026/0028374 A1
(43) Pub. Date: Jan. 29, 2026

(54) EPIDERMAL GROWTH FACTOR RECEPTOR INHIBITORS

(71) Applicant: FILAMON LTD, Camberwell VIC (AU)

(72) Inventors: Timothy MANN, Camberwell VIC (AU); Jun ZENG, Camberwell VIC (AU); Kieran SCOTT, Camberwell VIC (AU)

(21) Appl. No.: 19/355,164

(22) Filed: Oct. 10, 2025

Related U.S. Application Data
(63) Continuation of application No. PCT/AU2024/050616, filed on Jun. 13, 2024.

(30) Foreign Application Priority Data
Sep. 4, 2023 (AU) 2023902843

(51) Int. Cl. C07K 7/64; A61K 31/517; A61K 38/00; A61P 35/00; C07K 1/04; C07K 16/28; G01N 33/50

(52) U.S. Cl. CPC C07K 7/64; A61K 31/517; A61P 35/00; C07K 1/04; C07K 16/2863; G01N 33/5044; A61K 38/00; G01N 2500/04; G01N 2500/10

(57) ABSTRACT

The present disclosure generally relates to epidermal growth factor receptor (EGFR) inhibitors and methods of treating EGFR-dependent or associated diseases, disorders or conditions, such as cancer.

### CROSS-REFERENCE TO RELATED APPLICATIONS

This application is a continuation of International Patent Application No. PCT/AU2024/050947, filed Sep. 4, 2024, which claims priority to Australian Provisional Patent Application No. 2023902843, filed Sep. 4, 2023, the contents of which are incorporated herein by reference in their entirety.

### SEQUENCE LISTING

The instant application contains a Sequence Listing which has been submitted electronically in XML format and is hereby incorporated by reference in its entirety. Said XML file, created on Oct. 9, 2025, is named 770713_FBR9-008PCCON_ST26.xml and is 7,183 bytes in size.

### TECHNICAL FIELD

The present disclosure generally relates to epidermal growth factor receptor (EGFR) inhibitors and methods of treating EGFR-dependent or associated diseases, disorders or conditions, such as cancer.

### BACKGROUND

The epidermal growth factor receptor (EGFR) is a 170 kDa transmembrane protein that belongs to the ErbB family of tyrosine kinases¹. EGFR is overexpressed in multiple types of cancers and is the subject of inhibition in clinical treatments of non-small cell lung cancer (NSCLC), colorectal cancer (CRC), head and neck cancer and glioblastoma². In normal physiology, a ligand of EGFR such as epidermal growth factor (EGF), binds to the extracellular domain of the receptor causing dimerization of EGFR with another EGFR or any other receptor from the ErbB family. Dimerization initiates sequestration of EGFR and signal transduction resulting from the phosphorylation of its intracellular tyrosine kinase, activating key pathways RAS/RAF/MEK/ERK and the PI3K/AKT, as well as PKC, Src tyrosine kinases, PLC7 and STAT downstream signaling³. The aberrant activation of these pathways is crucial for the pathological cell proliferation, growth and migration seen in cancer and other EGFR-associated diseases.

Monoclonal antibodies such as cetuximab that target EGFR, block ligand activation and are a major class of clinically used inhibitor, primarily for the treatment of head and neck cancer and CRC. Cetuximab treatment is limited to a small subset of patients that do not harbour KRAS mutations⁴, as de novo resistance arises from KRAS, BRAF and NRAS genes that lead to aberrant MAPK signalling⁵. Further resistance can arise from the S492R mutation⁶, inhibiting cetixumab but not EGF ligand binding, or through mutations leading to ligand independent activation⁷.

Tyrosine kinase inhibitors (TKIs) are the second major class of EGFR inhibitor that are clinically used in the treatment of NSCLC. TKIs target the intracellular tyrosine kinase domain of EGFR, however resistance typically develops as this region is genetically unstable and prone to mutation⁸.

Accordingly, there remains a clinical need for additional EGFR inhibitors that overcome one or more of the drawbacks of prior art inhibitors.

### SUMMARY

The present disclosure is based, in part, on the discovery of a new class of EGFR inhibitor that bind to the extracellular domain of EGFR and initiate non-clathrin mediated endocytosis (non-CME) and lysosomal degradation of EGFR protein. The present disclosure provides cyclic peptides with potent and long-lasting inhibitory activity in relation to EGFR expression and signalling. The inventors have also shown that these cyclic peptides have an anti-cancer effect in vitro.

In a first aspect, the present disclosure provides a cyclic peptide comprising the sequence of the formula:

Xaa¹-Xaa²-Xaa³-Xaa⁴-Xaa⁵

in which:
Xaa¹ is F or 1NapA;
Xaa² is L or I;
Xaa³ is S or T;
Xaa⁴ is F or 2NapA; and
Xaa⁵ is R or K.

Suitably:
(a) Xaa¹ is 1NapA and Xaa⁴ is 2NapA;
(b) Xaa¹ is F and Xaa⁴ is 2NapA;
(c) Xaa¹ is F and Xaa⁴ is F; or
(d) Xaa¹ is 1NapA and Xaa⁴ is F.

In a second aspect, the present disclosure provides a cyclic peptide comprising the sequence of Formula I, or a pharmaceutically acceptable salt, solvate or prodrug thereof:

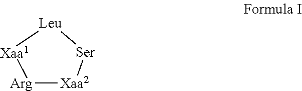

wherein:
Xaa¹ is selected from 1-naphthyl-alanine and phenylalanine; and
Xaa² is selected from 2-naphthyl-alanine and phenylalanine.

In particular examples of the above aspects, the cyclic peptide comprises the sequence of:

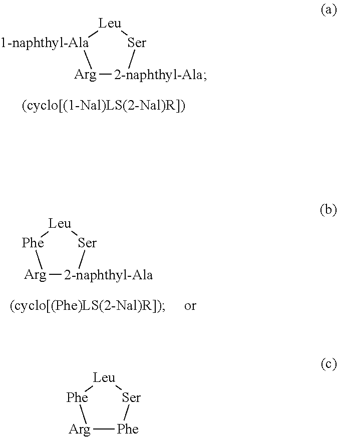

(cyclo[(Phe)LS(Phe)R]);
   or a pharmaceutically acceptable salt, solvate or prodrug thereof.

According to some examples of the aforementioned aspects, the cyclic peptide comprises the chemical structure of:

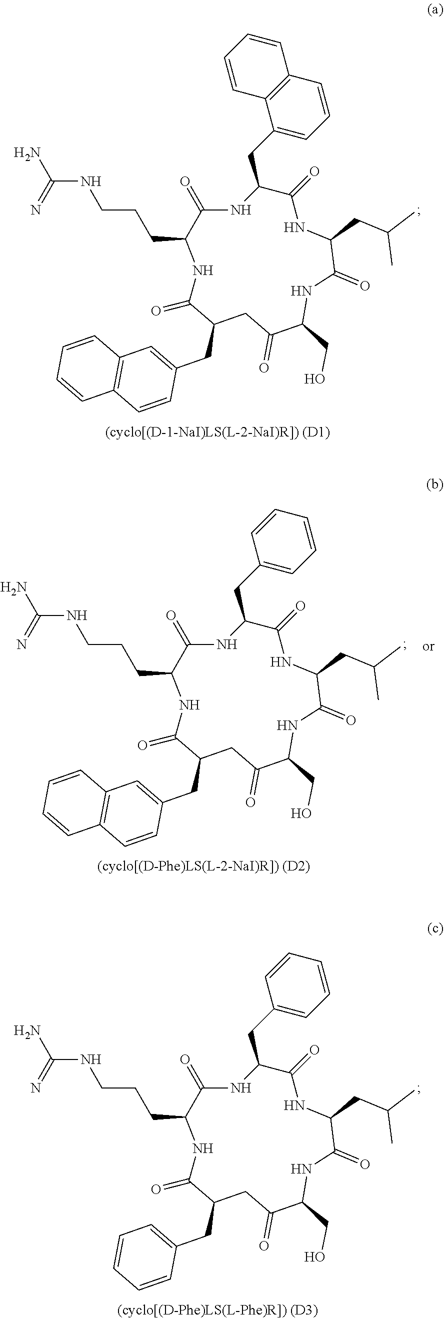 [SMILES: CC(C)C[C@@H]1NC(=O)[C@H](Cc2cccc3ccccc23)NC(=O)[C@H](CCCNC(=N)N)NC(=O)[C@H](Cc2ccc3ccccc3c2)CC(=O)[C@H](CO)NC1=O ; CC(C)C[C@@H]1NC(=O)[C@H](Cc2ccccc2)NC(=O)[C@H](CCCNC(=N)N)NC(=O)[C@H](Cc2ccc3ccccc3c2)CC(=O)[C@H](CO)NC1=O ; CC(C)C[C@@H]1NC(=O)[C@H](Cc2ccccc2)NC(=O)[C@H](CCCNC(=N)N)NC(=O)[C@H](Cc2ccccc2)CC(=O)[C@H](CO)NC1=O]

or a pharmaceutically acceptable salt, solvate or prodrug thereof.

Suitably, the cyclic peptide of the above aspects is in the form of an acetate salt.

In a third aspect, the present disclosure provides a pharmaceutical composition, comprising the cyclic peptide of any one of the preceding claims and a pharmaceutically acceptable carrier, diluent or excipient.

Suitably, the cyclic peptide of the first or second aspects or the pharmaceutical composition of the third aspect is for use in therapy.

Suitably, the cyclic peptide of the first or second aspects or the pharmaceutical composition of the third aspect is for use in treating, preventing or ameliorating an EGFR-related disease, disorder or condition.

In a fourth aspect, the present disclosure provides a method of treating, preventing or ameliorating an EGFR-related disease, disorder or condition in a subject, said method including the step of administering a therapeutically effective amount of the cyclic peptide of the first or second aspects or the pharmaceutical composition of the third aspect to the subject to thereby treat, prevent or ameliorate the EGFR-related disease, disorder or condition.

Suitably, the EGFR-related disease, disorder or condition is an EGFR-related cancer, such as prostate cancer, non-small cell lung cancer, pancreatic cancer, ovarian cancer, gastrointestinal cancer, rectal cancer, kidney cancer, liver cancer, gallbladder cancer, head and neck cancer, transitional cell carcinoma, squamous cell carcinoma, melanoma, glioblastoma, gliosarcoma, colorectal cancer, breast cancer, oesophageal cancer, bladder cancer, hepatocellular carcinoma, renal cell carcinoma, brain and central nervous system cancer, neuroendocrine cancer, lymphoma, multiple myeloma, or chronic lymphocytic leukaemia.

In a fifth aspect, the present disclosure provides a method of inhibiting EGFR in a cell, said method including the step of contacting the cell with an effective amount of the cyclic peptide of the first or second aspects or the pharmaceutical composition of the third aspect to thereby inhibit EGFR in the cell.

In a sixth aspect, the present disclosure provides a method of promoting internalisation and/or lysosomal degradation of EGFR in a cell, said method including the step of contacting the cell with an effective amount of the cyclic peptide of the first or second aspects or the pharmaceutical composition of the third aspect to thereby promote internalisation and/or lysosomal degradation of EGFR in the cell.

With respect to the fifth and sixth aspects, the cell is suitably a cancer cell.

In a seventh aspect, the present disclosure provides a method of reducing the viability and/or growth of a cancer cell, said method including the step of contacting the cancer cell with an effective amount of the cyclic peptide of first or second aspects or the pharmaceutical composition of the third aspect to thereby reduce the viability and/or growth of the cancer cell.

Referring to the fifth, sixth and seventh aspects, the cancer cell may be a prostate cancer cell, a non-small cell lung cancer cell, a pancreatic cancer cell, an ovarian cancer cell, a gastrointestinal cancer cell, a rectal cancer cell, a kidney cancer cell, a liver cancer cell, a gallbladder cancer cell, a head and neck cancer cell, a transitional cell carcinoma cell, a squamous cell carcinoma cell, a melanoma cell, a glioblastoma cell, a gliosarcoma cell, a colorectal cancer cell, a breast cancer cell, an oesophageal cancer cell, a bladder cancer cell, a hepatocellular carcinoma cell, a renal cell carcinoma cell, a brain and central nervous system cancer cell, a neuroendocrine cancer cell, a lymphoma cell, a multiple myeloma cell, or a chronic lymphocytic leukaemia cell. In certain examples, the cancer cell is an EGFR-related cancer cell.

In an eighth aspect, the present disclosure provides for the use of the cyclic peptide of the first or second aspects or the pharmaceutical composition of the third aspect in the manufacture of a medicament for use in therapy.

In a ninth aspect, the present disclosure provides for the use of the cyclic peptide of the first or second aspects or the pharmaceutical composition of the third aspect in the manufacture of a medicament for treating, preventing or ameliorating an EGFR-related disease, disorder or condition.

In a tenth aspect, the present disclosure provides a method of identifying, designing or producing an agent capable of interacting with or binding to a ligand binding domain of EGFR, said method including the steps of:
(a) contacting a cell expressing EGFR with a candidate agent, wherein the candidate agent is a derivative, variant or analogue of: (i) cyclo-((2-Nal)-Leu-Ser-(2-Nal)-Arg); or (ii) the cyclic peptide of the first or second aspects; and
(b) determining whether the candidate agent inhibits the binding of an EGFR ligand to EGFR of the cell.

In some examples, the EGFR ligand is EGF or hGIIA.

In certain examples, a ligand binding domain of EGFR comprises or is defined by one or more residues selected from amino acids Q8, G9, T10, S11, N12, K13, L14, T15, Q16, L17, H23, F24, L25, S26, L27, Q28, R29, M30, L41, E42, 143 and T44 of SEQ ID NO: 2.

In other examples, the method further includes the step of determining whether the candidate agent promotes lysosomal degradation of EGFR in the cell.

According to particular examples, the method further includes the step of isolating and/or purifying an agent that inhibits the binding of the EGFR ligand to EGFR. In such examples, the method can further include one or more of the steps: (a) formulating the isolated and/or purified agent into a pharmaceutically acceptable formulation; (b) sterilising the formulation; and filling the formulation into a container, such as a vial, an ampoule, a bag, a blister pack, a bottle, a cartridge, an injection needle, an injection syringe, a single dose container, a strip of multiple single dose containers, or a tube.

In an eleventh aspect, the present disclosure provides an agent capable of interacting with or binding to a ligand binding domain of EGFR, obtained by the method of the tenth aspect.

In a twelfth aspect, the present disclosure provides a method of manufacturing the cyclic peptide of the first or second aspects, wherein said method comprises solid phase synthesis.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1. (a), Amino acid sequence of human group IIA-secreted phospholipase A₂ (hGIIA) predicted to interact with EGF-binding site of EGFR shown in magenta; helices shown as red braids; β strands as green arrows; and loops as blue lines. Sequence from which FLM-c2 is derived shown with orange box. (b), Prostate cancer cell line PC-3 incubated with hGIIA (2 μg/mL) and FLM-c2 (100 μM) for 3 hours before lysing and pulldown with hGIIA antibody followed by detection with EGFR antibody. (c), molecular modelling of EGF (pink ribbon) and (d), FLM-c2 analogue (cyclo-(Phe-Leu-Ser-Phe-Arg)) (stick diagram. Yellow, carbon; blue, nitrogen; red oxygen) binding to EGFR. EGFR is depicted as a surface model (red, basic surfaces; blue, acidic surfaces, yellow; cysteine residues; green, hydrophobic surfaces; white neutral surfaces). (e) PC-3 cells co-transfected with Rab5-mCherry (red), EGFR-GFP (green) and treated with hGIIA/AF647 (purple) as well as c2 at 1 μM and 10 μM for 24 hours. EGF (25 ng/mL) and Lapatinib (5 μM) were used as control treatments. N=3, >5 cells per replicate.

FIG. 2. (a) Structure of FLM-c2 derivatives D1 (top), D2 (middle) and D3 (bottom) (b) Molecular modelling of compounds D1, D2 and D3. Representation of the molecules are as described in FIG. 1. (c) MTS assay of PC-3 cells treated with FLM-c2, D-1 D3 (0.1-250 μM). (d) AlphaLISA in vitro assay of EGF-EGFR interaction with the addition of FLM-c2, D1-D3 and erlotinib.

FIG. 3. (a) PC-3 cells treated with DMSO (0.5%), FLM-c2 (100 μM), D1-D3 (100 μM) or EGF (50 ng/mL) for 24 h, detected for EGFR and GAPDH. (b) PC-3 cells tagged with EGFR-EGFP and lysotracker treated with DMSO (0.5%) or D3 (100 μM) for 24 h.

FIG. 4. (a) PC-3 cells were treated with D1-D3 (100 μM) for 3, 16, 24 and 48 hours. EGFR and ß-Actin ratio was detected. (b) the EGFR:ß-Actin ratio was plotted after normalising to DMSO control.

FIG. 5: (a) A549 cells were treated with DMSO (0.5%), EGF (50 ng/mL), or D1-D3 (1-100 μM) for 16 hours, before staining with lysotracker (red), fixing and staining with EGFR antibody (green). (b) EGFR signal, (c) EGFR membrane signal, (d) lysotracker signal and (e) EGFR speckle lysotracker signal (bars SEM) were quantified (n=3, >20 cells per replicate). Scale bars 20 μm.

FIG. 6: A549 cells were treated with DMSO or D1-D3 (10-100 μM) for 16 hours before incubation with EGF (10 ng/mL) for 30 minutes, before cells were fixed and stained for pERK (green). Scale bars 20 μm, n=3, >20 cells per replicate.

FIG. 7: Cell viability of NSCLC cell lines H3255, HCC827 and H1975 and CRC cell lines HCT116, SW48, SW48G12V, HT29 and RKO following incubation with c2 or D1-D3 at 0.01-300 μM for 72 hours.

FIG. 8: Cell viability of NSCLC cell lines H1975 and H3255 after treatment with Cetuximab (0.01-100 μg/mL), U0126 chloroquine and D1 (0.001-300 μM) alone and in combination for 72 hours.

FIG. 9: Cell viability of SW48 and SW48G12V cells treated with Cetuximab (0.2-30 μg/mL) in combination with D1 (10-100 μM) for 72 hours.

Key to the Sequence Listing

| | |
|---|---|
| SEQ ID NO: 1 | Amino acid sequence for a reference human |
| | EGFR protein (including signal peptide) |
| SEQ ID NO: 2 | Amino acid sequence for a reference human |
| | EGFR protein (mature version without signal peptide) |
| SEQ ID NO: 3 | Amino acid sequence for a reference human |
| | EGF protein |

### DETAILED DESCRIPTION

### General

Throughout this specification, unless specifically stated otherwise or the context requires otherwise, reference to a single step, feature, composition of matter, group of steps or group of features or compositions of matter shall be taken to encompass one and a plurality (i.e. one or more) of those steps, features, compositions of matter, groups of steps or groups of features or compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant DNA, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

Throughout this specification, unless the context requires otherwise, the word “comprise”, or variations such as “comprises” or “comprising”, is understood to imply the inclusion of a stated step or element or integer or group of steps or elements or integers but not the exclusion of any other step or element or integer or group of elements or integers.

By “consisting essentially of”, in the context of an amino acid sequence, is meant the recited amino acid sequence together with an additional one, two or three amino acids at the N- or C-terminus. Moreover, in the context of a nucleotide sequence, the term “consisting essentially of”, is meant the recited nucleotide sequence together with an additional one, two or three nucleic acid residues or bases at the 5′ end or 3′ end thereof.

The term “and/or”, e.g., “X and/or Y” shall be understood to mean either “X and Y” or “X or Y” and shall be taken to provide explicit support for both meanings or for either meaning.

As used herein, the terms “approximately” and “about” refer to tolerances or variances associated with numerical values recited herein (e.g., ±0.1%, 0.5%, 1.0%, 5.0% or 10%). The extent of such tolerances and variances are well understood by persons skilled in the art. Typically, such tolerances and variances do not compromise the structure, function and/or implementation of the compositions and methods described herein.

All computer programs, algorithms, patent and scientific literature referred to herein is incorporated herein by reference.

For the present disclosure, the database accession number or unique identifier provided herein for a gene or protein, as well as the gene and/or protein sequence or sequences associated therewith, are incorporated by reference herein.

### Cyclic Peptide Inhibitors of EGFR

The inventors have surprisingly shown for the first time that derivatives of the cyclic peptide c2 can bind or interact with a ligand binding domain of EGFR and in doing so promote subsequent sequestration and lysosomal degradation of this receptor.

Accordingly, in one broad form, the present disclosure provides a cyclic peptide which is a variant, derivative or analogue of cyclo-((2-Nal)-Leu-Ser-(2-Nal)-Arg) (referred to herein as FLM-c2 or c2), cFLSYR or cFLSYK, that binds or interacts with a ligand binding domain on EGFR. For example, the cyclic peptide may be of the following formula:

Xaa¹-Xaa²-Xaa³-Xaa⁴-Xaa⁵

in which:
Xaa¹ is an aromatic and uncharged amino acid residue (inclusive of natural and unnatural amino acid residues), such as F or 1NapA;
Xaa² is L or I;
Xaa³ is S or T;
Xaa⁴ is an aromatic and uncharged amino acid residue (inclusive of natural and unnatural amino acid residues), such as F or 2NapA; and
Xaa⁵ is R or K.

In certain examples, Xaa² is L.

In some examples, Xaa³ is S.

In other examples, Xaa⁵ is R.

In particular examples, Xaa⁵ is K.

Suitably, Xaa² is L, Xaa³ is S and Xaa⁵ is R. In these examples, the cyclic peptide may be considered to be a derivative, analogue or variant of FLM-c2 or cFLSYR.

In alternative examples, Xaa² is L, Xaa³ is S and Xaa⁵ is K. For such examples, the cyclic peptide may be considered to be a derivative of cFLSYK.

For certain examples, Xaa¹ is 1NapA and Xaa⁴ is 2NapA. In some examples, Xaa¹ is 1NapA, Xaa² is L, Xaa³ is S, Xaa⁴ is 2NapA and Xaa⁵ is R. In alternative examples, Xaa¹ is 1NapA, Xaa² is L, Xaa³ is S, Xaa⁴ is 2NapA and Xaa⁵ is K.

According to other examples, Xaa¹ is F and Xaa⁴ is 2NapA. In some examples, Xaa¹ is F, Xaa² is L, Xaa³ is S, Xaa⁴ is 2NapA and Xaa⁵ is R. In alternative examples, Xaa¹ is F, Xaa² is L, Xaa³ is S, Xaa⁴ is 2NapA and Xaa⁵ is K.

Referring to various examples, Xaa¹ is F and Xaa⁴ is F. In some examples, Xaa¹ is F, Xaa² is L, Xaa³ is S, Xaa⁴ is 2NapA and Xaa⁵ is R. In alternative examples, Xaa¹ is F, Xaa² is L, Xaa³ is S, Xaa⁴ is 2NapA and Xaa⁵ is K.

In other examples, Xaa¹ is 1NapA and Xaa⁴ is F. In some examples, Xaa¹ is 1NapA, Xaa² is L, Xaa³ is S, Xaa⁴ is F and Xaa⁵ is R. In alternative examples, Xaa¹ is 1NapA, Xaa² is L, Xaa³ is S, Xaa⁴ is F and Xaa⁵ is K.

In one particular form, the present disclosure relates to a cyclic peptide comprising the sequence of Formula I, or a pharmaceutically acceptable salt, solvate or prodrug thereof:

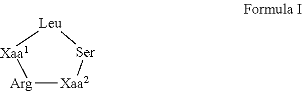

wherein:
Xaa¹ is selected from 1-naphthyl-alanine and phenylalanine; and
Xaa² is selected from 2-naphthyl-alanine and phenylalanine.

In some examples, Xaa¹ is selected from D-1-naphthyl-alanine and D-phenylalanine.

In certain examples, Xaa² is selected from L-2-naphthyl-alanine and L-phenylalanine.

By “protein” is meant an amino acid polymer. The amino acids may be natural or non-natural amino acids, D- or L-amino acids as are well understood in the art. As would be appreciated by the skilled person, the term “protein” also includes within its scope phosphorylated forms of a protein (i.e., a phosphoprotein) and/or glycosylated forms of a protein (i.e. a glycoprotein). A “peptide” is generally a protein having no more than fifty (50) amino acids. A “polypeptide” is generally a protein having more than fifty (50) amino acids.

Variants and analogues of the cyclic peptides described herein are also envisaged. As used herein, a protein, polypeptide or peptide “variant” or “analogue” shares a definable amino acid sequence relationship with a reference amino acid sequence. In particular examples, the reference amino acid sequence is that of Formula I. As such, the reference amino acid sequence may be the amino acid sequence of any one of cyclo[(1-Nal)LS(2-Nal)R], cyclo[(Phe)LS(2-Nal)R] or cyclo[(Phe)LS(Phe)R]. More particularly, the reference amino acid sequence may be the amino acid sequence of any one of cyclo[(D-1-Nal)LS(L-2-Nal)R], cyclo[(D-Phe)LS(L-2-Nal)R] or cyclo[(D-Phe)LS(L-Phe)R].

The “variant” or “analogue” protein, polypeptide or peptide may have one or a plurality of amino acids of the reference amino acid sequence deleted, inserted/added or substituted by different amino acids. It is well understood in the art that some amino acids may be substituted, inserted/added or deleted without changing the activity of the cyclic peptide (i.e., conservative substitutions). Accordingly, one or more of the residues of a cyclic peptide may be conservatively modified (e.g., by amino acid substitution or deletion) without altering the biological activity, function, or other desired property thereof, such as its affinity or its specificity for a ligand binding domain of EGFR.

In some examples, a variant of the cyclic peptide provided herein substantially retains the EGFR binding ability (i.e., a ligand binding domain thereof) of the unmodified or reference cyclic peptide. Thus, one or more amino acid residues within the cyclic peptide of the present disclosure can be deleted or replaced with other amino acid residues, such as those from the same side chain family, and the variant cyclic peptide can be tested for retained function (e.g., the ability to specifically bind a ligand binding domain of EGFR at high affinity) using the functional assays described herein.

Suitably, the cyclic peptide or the derivative, variant or analogue thereof is not cFLSYK, cFLSYR or c(2NapA)LS(2NapA)R (i.e., referred to as c2 or FLM-c2 herein). When used herein the term “cFLSYK” means “cyclic FLSYK”, “cFLSYR” means “cyclic FLSYR” and “c(2NapA)LS(2NapA)R” means “cyclic (2NapA)LS (2NapA)R”. The terms “2NapA”, “2-Nal” and “2-naphthyl-Ala” are abbreviations for 2-naphthylalanine. Similarly, the terms “1NapA”, “I-Nal” and “1-naphthyl-Ala” are abbreviations for 1-naphthylalanine. In particular examples, the cyclic peptide or the derivative, variant or analogue of the cyclic peptide is not FLM-c2. In other examples, the cyclic peptide or the derivative, variant or analogue of the cyclic peptide is not cFLSYK. In further examples, the cyclic peptide or the derivative, variant or analogue of the cyclic peptide is not cFLSYR.

The present disclosure also contemplates derivatives of the cyclic peptides described herein. As used herein, “derivatives” are molecules such as proteins, fragments or variants thereof that have been altered, for example, by conjugation or complexing with other chemical moieties, by post-translational modification (e.g., phosphorylation, acetylation and the like), modification of glycosylation (e.g., adding, removing or altering glycosylation), lipidation and/or inclusion of additional amino acid sequences as would be understood in the art.

Derivatives contemplated by the disclosure include, but are not limited to, modification to side chains, incorporation of unnatural amino acids and/or their derivatives during peptide, or protein synthesis and the use of crosslinkers and other methods which impose conformational constraints on the cyclic peptides of the disclosure. In this regard, the skilled person is referred to Chapter 15 of CURRENT PROTOCOLS IN PROTEIN SCIENCE, Eds. Coligan et al. (John Wiley & Sons NY 1995-2008) for more extensive methodology relating to chemical modification of proteins.

Further derivatives may include conjugates of the cyclic peptides. The term “conjugated” can be used in the context of the present disclosure to describe cyclic peptides disclosed herein that are conjugated to another compound or structure, such as a label or carrier molecule or protein. Accordingly, in one example, the cyclic peptides of the present disclosure are “conjugated”. Cyclic peptides of the disclosure may be modified via conjugation or complexing with other chemical moieties, by post-translational modification (e.g., phosphorylation, ubiquitination, glycosylation), chemical modification (e.g., cross-linking, acetylation, biotinylation, oxidation or reduction) and/or conjugation with labels (e.g., fluorophores, enzymes, radioactive isotopes) and/or other functional elements (e.g., a half-life extender, a therapeutic agent), as are known in the art.

Such derivatives, variants and analogues for use according to the present disclosure are suitably a functional cyclic peptide. By “functional cyclic peptide” is meant a peptide able to bind to EGFR, such as a ligand binding domain thereof, and thereby prevent or inhibit binding of an EGFR ligand, such as EGF, thereto. Such functional cyclic peptides also suitably promote or facilitate internalisation, sequestration and/or lysosomal degradation of EGFR in a cell. Determining whether a cyclic peptide derivative, variant or analogue is functional may be assessed by any method or means known in the art, such as those described herein.

Suitably, the cyclic peptide comprises the sequence of cyclo[(1-Nal)LS(2-Nal)R], as illustrated below:

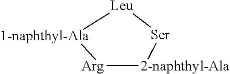

or a pharmaceutically acceptable salt, solvate or prodrug thereof.

For such examples, the cyclic peptide suitably comprises the chemical structure of:

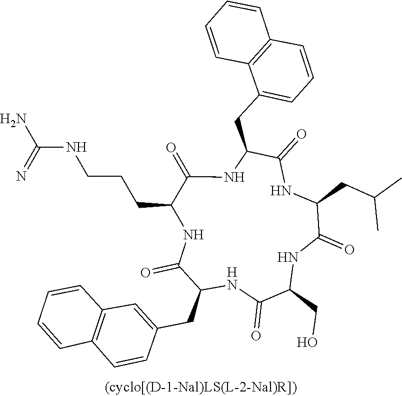

or a pharmaceutically acceptable salt, solvate or prodrug thereof.

Suitably, the cyclic peptide comprises the sequence of cyclo[(Phe)LS(2-Nal)R] or cyclo[FLS(2-Nal)R], as illustrated below:

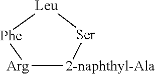

or a pharmaceutically acceptable salt, solvate or prodrug thereof.

For these examples, the cyclic peptide suitably comprises the chemical structure of:

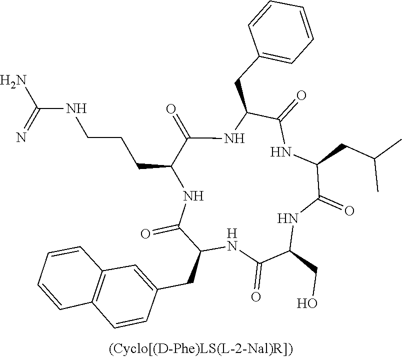

or a pharmaceutically acceptable salt, solvate or prodrug thereof.

In various examples, the cyclic peptide comprises the sequence of cyclo[(Phe)LS(Phe)R] or cyclo[FLSFR], as illustrated below:

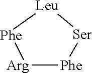

or a pharmaceutically acceptable salt, solvate or prodrug thereof.

According to such examples, the cyclic peptide suitably comprises the chemical structure of:

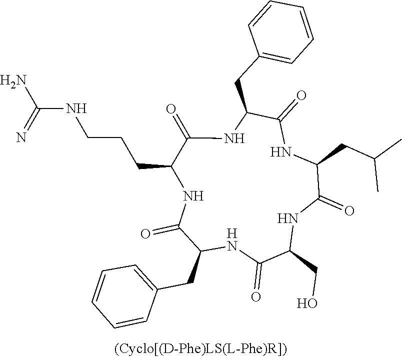

or a pharmaceutically acceptable salt, solvate or prodrug thereof.

In various examples, the cyclic peptide comprises the sequence of cyclo[(1-Nal)LS(2-Nal)K] or cyclo[(D-1-Nal)LS(L-2-Nal)K], or a pharmaceutically acceptable salt, solvate or prodrug thereof.

In other examples, the cyclic peptide comprises the sequence of cyclo[(Phe)LS(L-2-Nal)K], cyclo[FLS(L-2-Nal)K] or cyclo[(D-Phe)LS(L-2-Nal)K], or a pharmaceutically acceptable salt, solvate or prodrug thereof.

In certain examples, the cyclic peptide comprises the sequence of cyclo[(Phe)LS(Phe)K], cyclo[FLSFK], cyclo[(D-Phe)LS(L-Phe)K], or a pharmaceutically acceptable salt, solvate or prodrug thereof.

According to certain examples, the cyclic peptide described herein binds or interacts with a ligand binding domain on EGFR. The ligand binding domain of EGFR may comprise or be defined by one or more residues selected from amino acids Q8, G9, T10, S11, N12, K13, L14, T15, Q16, L17, H23, F24, L25, 526, L27, Q28, R29, M30, L41, E42, 143 and T44 of an EGFR protein (e.g., a mature EGFR protein), such as that set forth in SEQ ID NO: 2. More particularly, the ligand binding domain of EGFR suitably comprises or is defined by one or more residues selected from amino acids L17, H23, F24, L25, S26, L27, Q28, R29, M30, L41, E42, 143 and T44 of the EGFR protein set forth in SEQ ID NO: 2.

Suitably, the cyclic peptide provided herein specifically binds to EGFR or binds to EGFR with high affinity. As used herein, the term “binds” refers to the interaction of the cyclic peptide with EGFR and means that the interaction is dependent upon the presence of a particular structure (e.g., a binding site having a particular amino acid sequence) on EGFR that is recognised by the cyclic peptide. For example, and by virtue of the binding site, the cyclic peptide recognizes and binds to a specific target protein (e.g., EGFR) rather than to molecules or proteins generally.

As used herein, the term “specifically binds” shall be taken to mean that the binding interaction between a cyclic peptide disclosed herein and a target protein described herein (e.g., EGFR, or more particularly a ligand binding domain thereof) is dependent on detection of the target protein by the cyclic peptide. Accordingly, the cyclic peptide preferentially binds or recognizes the target protein even when present in a mixture of other molecules, proteins, nucleic acids or organisms.

As used herein, the terms “high affinity” and “relatively high affinity” are used interchangeably herein and refer to a binding affinity between a cyclic peptide and the target protein of interest (e.g., EGFR) with a K_{D} of at least about 10⁻⁵ M, more particularly at least about 10⁻⁶ M, more particularly at least about 10⁻⁷ M, even more particularly at least about 10⁻⁸ M and yet even more particularly between about 10⁻⁷ M to about 10⁻¹⁰ M.

As used herein, the terms “low affinity” and “relatively low affinity” are used interchangeably herein and refer to a binding affinity between a cyclic peptide and a protein with a K_{D} of less than about 10⁻⁵ M, preferably less than about 10⁻⁴ M, more preferably less than about 10⁻³ M and even more preferably between about 10⁻² M to about 10⁻⁴ M.

The determination of such affinity may be conducted under standard competitive binding immunoassay procedures, as are known in the art, such as electrophoretic shift assay (EMSA), ELISA and surface plasmon resonance (SPR).

Suitably, the cyclic peptide described herein at least partly inhibits or blocks the interaction or binding of an EGFR ligand to EGFR. By “EGFR ligand” is meant a molecule, more particularly a polypeptide, that binds to an EGF receptor, preferably at a ligand binding domain thereof (e.g., the ligand binding domain defined by one or more of residues 8-17, 23-30 and 41-44 of SEQ ID NO: 2). Non-limiting examples of EGFR ligands include EGF, hGIIA, transforming growth factor α, amphiregulin, heparin-binding EGF, vaccinia virus growth factor, gp30, epiregulin and heregulin. Those skilled in the art can identify other EGF ligands using procedures well-known in the art. In particular examples, the EGFR ligand is epidermal growth factor (EGF) or hGIIA. More particularly, the EGFR ligand suitably is or comprises EGF.

In view of the foregoing, the cyclic peptides of the present disclosure may be considered to be EGFR inhibitors. The term “inhibitor” as used herein refers to a molecule having the ability to inhibit a biological function and/or expression of a target polypeptide, such as EGFR. The term “selective inhibition” or “selectively inhibit” refers to the cyclic peptide's ability to preferentially reduce the target polypeptide's expression and/or signalling activity, such as a kinase activity or binding activity, as compared to off-target signalling activity, via direct or indirect interaction with the target polypeptide.

Inhibition of EGFR activity and/or signalling by the cyclic peptides may be assessed by any means known in the art. By way of example, EGFR signalling and/or kinase activity may be measured using various well known methods, such as measuring the autophosphorylation of the receptor at any of tyrosine residues Y1068, Y1148, and Y1173 (Downward et al., Nature 311:483-5, 1984) and/or phosphorylation of natural or synthetic substrates. Phosphorylation can be detected using well known methods such as an ELISA assay or a western blot using a phosphotyrosine specific antibody. A level of EGFR activity and/or signalling may also be assessed indirectly by measuring a level of activation or signalling of one or more downstream signalling pathways, such as the MAPK pathway (e.g., Ras/Raf/Mek/Erk signalling), the PI3K/AKT pathway, the PKC pathway, a Src tyrosine kinase pathway, the PLC7 pathway and the STAT pathway. Commercially available EGFR kinase assays are also well known in the art (e.g., luminescent kinase assays that measure ADP formed from ATP in a kinase reaction). Levels of EGFR activity and/or signalling may also be assessed by detecting changes in EGFR expression levels following administration of a cyclic peptide of the present disclosure. The expression level of EGFR can be detected by methods known in the art, for example, immunohistochemistry, PCR, RT-PCR, in situ hybridization, Southern blot, Western blot, Northern blot, spectrophotometry, ELISA, and the like. Relative levels of cellular localisation of EGFR (e.g., extracellular and intracellular levels) may also be determined in assessing levels of EGFR activity and/or signalling.

The cyclic peptides of the present disclosure may block or inhibit binding of an EGFR ligand, such as EGF, to EGFR, such as human EGFR set forth in SEQ ID NO: 1 or 2. It is further envisaged that the present disclosure encompasses both partial and complete blocking/inhibition of EGFR by the cyclic peptides. Suitably, the cyclic peptides of the present disclosure may block or inhibit binding of an EGFR ligand to EGFR with an IC50 value of less than about 1×10⁵ M, less than about 1×10⁶ M, less than about 1×10⁷ M, less than about 1×10⁸ M, less than about 1×10⁹ M, less than about 1×10¹⁰ M, less than about 1×10¹¹ M, or less than about 1×10¹² M. IC50 may be assessed by any means known in the art, such as in a cell viability assay employing an EGFR-dependent cell line (e.g., an EGFR-dependent cancer cell line, such as PC-3) or an in vitro competition assay (e.g., AlphaLiSA® assay). Exemplary cyclic peptides may block EGFR ligand binding to EGFR with an IC50 value between about 1 μM to about 500 μM (e.g., about 1, 2, 3, 4, 5, 6, 7, 8, 9, 10, 20, 30, 40, 50, 60, 70, 80, 90, 100, 120, 140, 160, 180, 200, 220, 240, 260, 280, 300, 320, 340, 360, 380, 400, 420, 440, 460, 480 or 500 μM or any range therein), more particularly between about 100 μM to about 400 μM or even more particular between about 200 μM to about 300 μM. The cyclic peptides herein may block or reduce EGF (or another EGFR ligand) binding to EGFR and/or EGFR activity, such as EGFR kinase activity and EGFR-dependent signalling, by at least about 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% when compared to binding of EGF to EGFR in the absence of the cyclic peptide described herein using the same assay conditions.

The cyclic peptides of the present disclosure suitably facilitate or promote internalisation, sequestration and/or lysosomal degradation of EGFR in a cell. In particular examples, the cyclic peptides facilitate or promote non-clathrin-mediated endocytosis (non-CME) of EGFR in a cell. To this end, the cyclic peptides suitably decrease or reduce an expression level, such as a cell surface expression level, of EGFR in a cell. Suitably, the cyclic peptides decrease or reduce the expression level of EGFR in the cell for a time period of at least about 6 hours (e.g., 6, 12, 18, 24, 30, 36, 42, 48, 54, 60, 72 hours or any range therein), at least about 12 hours, at least about 24 hours or at least about 48 hours.

Recently, attention has been directed towards pharmaceutically modulating EGFR internalisation. Upon binding to a ligand, EGFR is either internalised via clathrin-mediated endocytosis (CME) or non-clathrin-mediated endocytosis (non-CME)⁹. CME leads to EGFR recycling back to the plasma membrane and subsequent sustained proliferative signalling, whereas non-CME leads EGFR to the lysosome for degradation¹⁰. It is thought that the pathway of internalisation is dictated by the affinity of the ligand that binds EGFR, as well as its concentration. Combination therapy of monoclonal antibodies futuximab and modotuximab known as Sym004 at high concentrations (3-30 μg/mL) can initiate lysosomal degradation of EGFR¹¹. Recently, cetuximab was linked with a glycoprotein to create a compound known as a lysosome targeting chimera (LYTAC) which also initiates lysosomal degradation of EGFR¹². An analogue of the naturally occurring 23-hydroxybetulinic acid known as DBPA also can initiate EGFR degradation¹³.

Accordingly, terms such as “higher”, “increased” and “greater” as used herein refer to an elevated level of internalisation, sequestration and/or lysosomal degradation of EGFR in a cell, such as in a cancer cell, when compared to a control or reference level or amount (e.g., a cell not contacted with the cyclic peptide). The level of internalisation, sequestration and/or lysosomal degradation of EGFR may be relative or absolute (i.e., relatively or absolutely higher, increased or greater). In some examples, the level of internalisation, sequestration and/or lysosomal degradation of EGFR is higher, increased or greater if its level is more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400% or at least about 500% above the level of internalisation, sequestration and/or lysosomal degradation of EGFR in a control or reference cell in the absence of the cyclic peptide.

Similarly, the terms, “lower”, “reduced” and “decreased”, as used herein refer to a lower amount or level of EGFR protein expression and/or activity in a cell, such as in a cancer cell, when compared to a control or reference level or amount (e.g., a cell not contacted with the cyclic peptide). The activity level and/or the expression level of EGFR may be relative or absolute (i.e., relatively or absolutely lower, reduced or decreased). In some examples, the activity level and/or the expression level of EGFR is lower, reduced or decreased if its level is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level of expression or activity of EGFR in a control or reference cell in the absence of the cyclic peptide.

The cyclic peptides of the present disclosure may be utilized per se or in the form of a pharmaceutically acceptable salt, ester, amide, solvate, prodrug, or isomer, as appropriate. For example, the cyclic peptide may be provided as a pharmaceutically acceptable salt. If used, a salt of the cyclic peptide should be both pharmacologically and pharmaceutically acceptable, but non-pharmaceutically acceptable salts may conveniently be used to prepare the free active compound or pharmaceutically acceptable salts thereof and are not excluded from the scope of this disclosure. Such pharmacologically and pharmaceutically acceptable salts can be prepared by reaction of the cyclic peptide with an organic or inorganic acid, using standard methods detailed in the literature.

The cyclic peptides disclosed herein, or of a formula disclosed herein, may be in the form of any pharmaceutically acceptable salt. The term “pharmaceutically acceptable salts” refers to salts prepared from pharmaceutically acceptable non-toxic bases or acids including inorganic or organic bases and inorganic or organic acids. Examples of salts derived from inorganic bases can include aluminium, ammonium, calcium, copper, ferric, ferrous, lithium, magnesium, manganic salts, manganous, potassium, sodium, zinc, and the like. Exemplary salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the cyclic peptides disclosed herein, or of a formula disclosed herein, are basic, acid addition salts may be prepared from pharmaceutically acceptable non-toxic acids, including inorganic and organic acids. Such acids include acetic, benzenesulfonic, benzoic, camphorsulfonic, carboxylic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, TFA, and the like. Acid addition salts of the peptides disclosed herein, or of a formula disclosed herein, are prepared in a suitable solvent from the peptide and an excess of an acid, such as hydrochloric, hydrobromic, sulfuric, phosphoric, acetic, TFA, citric, tartaric, maleic, succinic or methanesulfonic acid.

In particular examples, the cyclic peptide is in the form of an acetate salt.

The cyclic peptides disclosed herein can also exist in solvated forms, including solvates of the free peptide or solvates of a salt of the compound, as well as unsolvated forms. The term “solvate” is used herein to describe a molecular complex comprising the compound of the invention and one or more pharmaceutically acceptable solvent molecules, for example, ethanol. The term “hydrate” is employed when said solvent is water. It is to be understood that all polymorphs, including mixtures of different polymorphs, are included within the scope of the cyclic peptides described herein.

Esters of the cyclic peptides according to the present disclosure may be prepared through functionalization of hydroxyl and/or carboxyl groups that may be present within the compound. Amides and prodrugs may also be prepared using techniques known to those skilled in the art. For example, amides may be prepared from esters, using suitable amine reactants, or they may be prepared from an anhydride or an acid chloride by reaction with ammonia or a lower alkyl amine. Moreover, esters and amides of compounds of the invention can be made by reaction with a carbonylating agent (e.g., ethyl formate, acetic anhydride, methoxyacetyl chloride, benzoyl chloride, methyl isocyanate, ethyl chloroformate, methanesulfonyl chloride) and a suitable base (e.g., 4-dimethylaminopyridine, pyridine, triethylamine, potassium carbonate) in a suitable organic solvent (e.g., tetrahydrofuran, acetone, methanol, pyridine, N,N-dimethylformamide) at a temperature of 0° C. to 60° C.

As used herein, the term “prodrug” means a derivative of a compound that can hydrolyze, oxidize, or otherwise react under biological conditions (in vitro or in vivo) to provide a cyclic peptide described herein. Prodrugs may become active upon such reaction under biological conditions, or they may have activity in their unreacted forms. Examples of prodrugs contemplated for the present disclosure include, but are not limited to, analogues or derivatives of compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds described herein that comprise —NO, —NO₂, —ONO, or —ONO₂ moieties, Prodrugs can typically be prepared using well-known methods, such as those described by BURGER'S MEDICINAL CHEMISTRY AND DRUG DISCOVERY (1995) 172-178, 949-982 (Manfred E. Wolff ed., 5th ed).

According to various examples, the cyclic peptides described herein are for use in therapy. More particularly, the cyclic peptides described herein are suitably for use in treating, preventing or ameliorating an EGFR-related disease, disorder or condition, such as a cancer.

### EGFR and EGF

The terms “epidermal growth factor receptor” or “EGFR” as used herein generally refer to the Epidermal Growth Factor Receptor, also known as ErbB1 or HER1 (Ullrich et al., Nature 309:418-425, 1984), which is a 170 kDa transmembrane glycoprotein encoded by the c-ErbB1 protooncogene. EGFR is a member of the human epidermal growth factor receptor (HER) family of Receptor Tyrosine Kinases (RTKs) which also includes HER2(ErbB2), HER3(ErbB3) and HER4(ErbB 4). EGFR signalling is initiated by ligand binding, followed by initiation of a signalling cascade by inducing conformational changes, homo- or heterodimerization of the receptor with other ErbB family members, and trans-autophosphorylation of the receptor, among others (see Ferguson et at, Annu Rev biophysics, 37: 353-73, 2008), ultimately affecting a variety of cellular functions (e.g., cell proliferation and survival). Increased expression of EGFR, or its kinase activity, is associated with a range of human cancers, such as glioma, lung cancer, colorectal cancer, head and neck cancer, breast cancer, ovarian cancer, prostate cancer, cervical cancer, and the like.

Protein sequences of EGFR are publicly available (e.g., P00533). An exemplary amino acid sequence of human EGFR is set forth in SEQ ID NO: 1. The present disclosure also envisages naturally-occurring variants of EGFR. Such variants include the well-known EGFRvIII and other alternatively spliced variants (e.g., as identified by UniProt Accession numbers P00533-1, P00533-2, P00533-3, P00533-4), or other variants (e.g., GLN-98, ARG-266, LYS-521, ILE-674, GLY-962, and PRO-988), inclusive of disease- (e.g., cancer) and drug resistance-based EGFR variants, known in the art. The present disclosure also encompasses the mature version of the EGFR protein (i.e., minus the signal peptide-residues 1-24 of SEQ ID NO: 1), such as that provided in SEQ ID NO: 2. Thus, the EGFR amino acid sequence may be a protein which is at least 70%, 75%, 80%, 85%, 90%, 95%, 96%, 97%, 98%, 99%, 99.5% or 100% identical to SEQ ID NO:1 or SEQ ID NO:2 or a fragment or derivative thereof.

EGFR protein
(SEQ ID NO: 1)
MRPSGTAGAALLALLAALCPASRALEEKKVCQGTSNKLTQLGTFEDHFLS
LQRMENNCEVVLGNLEITYVQRNYDLSFLKTIQEVAGYVLIALNTVERIP
LENLQIIRGNMYYENSYALAVLSNYDANKTGLKELPMRNLQEILHGAVRF
SNNPALCNVESIQWRDIVSSDELSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLS
INATNIKHFKNCTSISGDLHILPVAFRGDSFTHTPPLDPQELDILKTVKE
ITGELLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFMRRRHIVRKRTLRRLLQERELVEPLTPSGEAPN
QALLRILKETEFKKIKVLGSGAFGTVYKGLWIPEGEKVKIPVAIKELREA
TSPKANKEILDEAYVMASVDNPHVCRLLGICLTSTVQLITQLMPFGCLLD
YVREHKDNIGSQYLLNWCVQIAKGMNYLEDRRLVHRDLAARNVLVKTPQH
VKITDFGLAKLLGAEEKEYHAEGGKVPIKWMALESILHRIYTHQSDVWSY
GVTVWELMTFGSKPYDGIPASEISSILEKGERLPQPPICTIDVYMIMVKC
WMIDADSRPKFRELIIEFSKMARDPQRYLVIQGDERMHLPSPTDSNFYRA
LMDEEDMDDVVDADEYLIPQQGFFSSPSTSRTPLLSSLSATSNNSTVACI
DRNGLQSCPIKEDSFLQRYSSDPTGALTEDSIDDTFLPVPEYINQSVPKR
PAGSVQNPVYHNQPLNPAPSRDPHYQDPHSTAVGNPEYLNTVQPTCVNST
EDSPAHWAQKGSHQISLDNPDYQQDFFPKEAKPNGIFKGSTAENAEYLRV
APQSSEFIGA
Mature EGFR protein
(SEQ ID NO: 2)
LEEKKVCQGTSNKLTQLGTFEDHFLSLQRMENNCEVVLGNLEITYVQRNY
DLSFLKTIQEVAGYVLIALNTVERIPLENLQIIRGNMYYENSYALAVLSN
YDANKTGLKELPMRNLQEILHGAVRESNNPALCNVESIQWRDIVSSDFLS
NMSMDFQNHLGSCQKCDPSCPNGSCWGAGEENCQKLTKIICAQQCSGRCR
GKSPSDCCHNQCAAGCTGPRESDCLVCRKERDEATCKDTCPPLMLYNPTT
YQMDVNPEGKYSFGATCVKKCPRNYVVTDHGSCVRACGADSYEMEEDGVR
KCKKCEGPCRKVCNGIGIGEFKDSLSINATNIKHFKNCTSISGDLHILPV
AFRGDSFTHTPPLDPQELDILKTVKEITGFLLIQAWPENRTDLHAFENLE
IIRGRTKQHGQFSLAVVSLNITSLGLRSLKEISDGDVIISGNKNLCYANT
INWKKLFGTSGQKTKIISNRGENSCKATGQVCHALCSPEGCWGPEPRDCV
SCRNVSRGRECVDKCNLLEGEPREFVENSECIQCHPECLPQAMNITCTGR
GPDNCIQCAHYIDGPHCVKTCPAGVMGENNTLVWKYADAGHVCHLCHPNC
TYGCTGPGLEGCPTNGPKIPSIATGMVGALLLLLVVALGIGLFMRRRHIV
RKRTLRRLLQERELVEPLTPSGEAPNQALLRILKETEFKKIKVLGSGAFG
TVYKGLWIPEGEKVKIPVAIKELREATSPKANKEILDEAYVMASVDNPHV
CRLLGICLTSTVQLITQLMPFGCLLDYVREHKDNIGSQYLLNWCVQIAKG
MNYLEDRRLVHRDLAARNVLVKTPQHVKITDFGLAKLLGAEEKEYHAEGG
KVPIKWMALESILHRIYTHQSDVWSYGVTVWELMTFGSKPYDGIPASEIS
SILEKGERLPQPPICTIDVYMIMVKCWMIDADSRPKFRELIIEFSKMARD
PQRYLVIQGDERMHLPSPTDSNFYRALMDEEDMDDVVDADEYLIPQQGFF
SPSTSRTPLLSSLSATSNNSTVACIDRNGLQSCPIKEDSFLQRYSSDPTG
ALTEDSIDDTFLPVPEYINQSVPKRPAGSVQNPVYHNQPLNPAPSRDPHY
QDPHSTAVGNPEYLNTVQPTCVNSTEDSPAHWAQKGSHQISLDNPDYQQD
FFPKEAKPNGIFKGSTAENAEYLRVAPQSSEFIGA

“Epidermal growth factor” (EGF) as used herein refers to the well-known growth factor, inclusive of variants and fragments thereof that maintain biological activity of EGF, encoded by the EGF gene that stimulates cell growth, proliferation, and differentiation by binding to its EGFR receptor. Protein sequences of EGF are publicly available (e.g., P01133-1, P01133-2, P01133-3). An exemplary amino acid sequence of human EGF is set forth in SEQ ID NO: 3. Thus, the EGF amino acid sequence may be a protein which is at least 70%, 75%, 80%, 85%, 90%, 95%, 96%, 97%, 98%, 99%, 99.5% or 100% identical to SEQ ID NO:3 or a fragment or derivative thereof.

EGF protein
(SEQ ID NO: 3)
MLLTLIILLPVVSKFSFVSLSAPQHWSCPEGTLAGNGNSTCVGPAPFLIF
SHGNSIFRIDTEGTNYEQLVVDAGVSVIMDFHYNEKRIYWVDLERQLLQR
VFLNGSRQERVCNIEKNVSGMAINWINEEVIWSNQQEGIITVTDMKGNNS
HILLSALKYPANVAVDPVERFIFWSSEVAGSLYRADLDGVGVKALLETSE
KITAVSLDVLDKRLFWIQYNREGSNSLICSCDYDGGSVHISKHPTQHNLF
AMSLFGDRIFYSTWKMKTIWIANKHTGKDMVRINLHSSFVPLGELKVVHP
LAQPKAEDDTWEPEQKLCKLRKGNCSSTVCGQDLQSHLCMCAEGYALSRD
RKYCEDVNECAFWNHGCTLGCKNTPGSYYCTCPVGFVLLPDGKRCHQLVS
CPRNVSECSHDCVLTSEGPLCFCPEGSVLERDGKTCSGCSSPDNGGCSQL
CVPLSPVSWECDCFPGYDLQLDEKSCAASGPQPFLLFANSQDIRHMHFDG
TDYGTLLSQQMGMVYALDHDPVENKIYFAHTALKWIERANMDGSQRERLI
EEGVDVPEGLAVDWIGRRFYWTDRGKSLIGRSDLNGKRSKIITKENISQP
RGIAVHPMAKRLFWTDTGINPRIESSSLQGLGRLVIASSDLIWPSGITID
FLTDKLYWCDAKQSVIEMANLDGSKRRRLTQNDVGHPFAVAVFEDYVWFS
DWAMPSVMRVNKRTGKDRVRLQGSMLKPSSLVVVHPLAKPGADPCLYQNG
GCEHICKKRLGTAWCSCREGFMKASDGKTCLALDGHQLLAGGEVDLKNQV
TPLDILSKTRVSEDNITESQHMLVAEIMVSDQDDCAPVGCSMYARCISEG
EDATCQCLKGFAGDGKLCSDIDECEMGVPVCPPASSKCINTEGGYVCRCS
EGYQGDGIHCLDIDECQLGEHSCGENASCTNTEGGYTCMCAGRLSEPGLI
CPDSTPPPHLREDDHHYSVRNSDSECPLSHDGYCLHDGVCMYIEALDKYA
CNCVVGYIGERCQYRDLKWWELRHAGHGQQQKVIVVAVCVVVLVMLLLLS
LWGAHYYRTQKLLSKNPKNPYEESSRDVRSRRPADTEDGMSSCPQPWFVV
IKEHQDLKNGGQPVAGEDGQAADGSMQPTSWRQEPQLCGMGTEQGCWIPV
SSDKGSCPQVMERSFHMPSYGTQTLEGGVEKPHSLLSANPLWQQRALDPP
HQMELTQ

Terms used generally herein to describe sequence relationships between respective nucleotides or polypeptides include “sequence identity”, “percentage of sequence identity” and “substantial identity”. Because respective nucleotides or polypeptides may each comprise (i) only one or more portions of a complete nucleotide or polypeptide sequence that are shared by the nucleotides or amino acids, and (ii) one or more portions which are divergent between the nucleotides or amino acids, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, WI, USA, incorporated herein by reference) or by inspection and the best alignment (i.e. resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, U) or amino acid residue occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, California, USA).

### Pharmaceutical Compositions

Further aspects of the present disclosure provide a composition comprising a cyclic peptide described herein, and one or more pharmaceutically acceptable carriers, diluents or excipients.

By “pharmaceutically-acceptable carrier, diluent or excipient” is meant a solid or liquid filler, diluent or encapsulating substance that may be safely used in systemic administration. Depending upon the particular route of administration, a variety of carriers well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water,

A useful reference describing pharmaceutically acceptable carriers, diluents and excipients is Remington's Pharmaceutical Sciences (Mack Publishing Co. NJ. USA, 1991), which is incorporated herein by reference.

According to some examples, the present composition is in the form of a therapeutic composition. A therapeutically effective amount of a composition comprising a cyclic peptide may be administered in a single dose, or in several doses, for example daily, during a course of treatment. However, the frequency of administration is dependent on the preparation applied, the subject being treated, the severity of the disease, disorder or condition (e.g., cancer), and the manner of administration of the therapy or composition.

Any safe route of administration may be employed for administering a cyclic peptide and pharmaceutical composition described herein. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be achieved by coating the same, for example, with hydrophobic polymers including acrylic resins, waxes, higher aliphatic alcohols, polylactic and polyglycolic acids and certain cellulose derivatives such as hydroxypropylmethyl cellulose, in addition, the controlled release may be achieved by using other polymer matrices, liposomes and/or microspheres. In particular examples, the composition is capable of being or configured or adapted to be administered orally to a subject in need thereof. For such examples, the composition is suitably enterically coated.

Compositions of the present disclosure suitable for oral or parenteral administration may be presented as discrete units such as capsules, sachets or tablets each containing a pre-determined amount of one or more therapeutic agents of the disclosure, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more therapeutic agents as described above with the carrier which constitutes one or more necessary ingredients. In general, the compositions are prepared by uniformly and intimately admixing the therapeutic agents of the disclosure with liquid carriers or finely divided solid carriers or both, and then, if necessary, shaping the product into the desired presentation.

The above compositions may be administered in a manner compatible with the dosage formulation, and in such an amount as is effective to prophylactically and/or therapeutically treat EGFR-associated or EGFR-dependent diseases, disorders or conditions and/or alleviate symptoms associated therewith. The dose administered to a patient, in the context of the present disclosure, should be sufficient to achieve a beneficial response in a patient over time such as a complete or partial response or stable disease (e.g., little or no disease progression). The quantity of the therapeutic agent(s) to be administered may depend on the subject to be treated inclusive of the age, sex, weight and general health condition thereof. In this regard, precise amounts of the therapeutic agent(s) required to be administered will depend on the judgement of the clinician. The total dose required for each treatment may be administered by multiple doses or in a single dose. In any event, suitable dosages of the therapeutic agents described herein may be readily determined by those skilled in the art. Such dosages may be in the order of nanograms to milligrams of the therapeutic agents of the disclosure.

Suitably, the composition further comprises one or more further or additional therapeutic agents, such as an anti-cancer agent, inclusive of those described herein.

According to various examples, the composition described herein is for use in therapy. More particularly, the composition described herein is suitably for use in treating, preventing or ameliorating an EGFR-related disease, disorder or condition, such as a cancer.

### Methods of Treatment

The present inventors have further demonstrated that the cyclic peptides described herein can inhibit proliferation and/or promote cell death in EGFR-dependent cancer cell lines. These cyclic peptides therefore offer promise as potential therapies for EGFR-dependent diseases, disorders and conditions, such as cancer.

Accordingly, in one broad form, the present disclosure provides a method of treating, preventing or ameliorating an EGFR-related disease, disorder or condition in a subject, said method including the step of administering a therapeutically effective amount of the cyclic peptide or the pharmaceutical composition described herein to the subject to thereby treat, prevent or ameliorate the EGFR-dependent disease, disorder or condition.

In another form, the present disclosure provides for the use of a cyclic peptide or a pharmaceutical composition described herein in the manufacture of a medicament for use in therapy.

In a related form, the present disclosure provides for the use of a cyclic peptide or a pharmaceutical composition described herein in the manufacture of a medicament for treating, preventing or ameliorating an EGFR-related disease, disorder or condition, such as a cancer.

As used herein, the terms “treating”, “treat”, “treatment” or “therapy” and variations thereof, refer to clinical intervention designed to alter the natural course of the individual or cell being treated during the course of clinical pathology. Desirable effects of treatment include decreasing the rate of disease progression, ameliorating or palliating the disease state, and remission or improved prognosis.

As used herein, the terms “prevent”, “prevented”, or “preventing”, refer to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. These terms also include within their scope preventing the disease or condition from occurring in a subject which may be predisposed to the disease or condition but has not yet been diagnosed as having it. The term “ameliorate” as used herein, unless otherwise specified, means to eliminate, delay, or reduce the prevalence or severity of symptoms associated with a disease, disorder or condition.

The term “therapeutically effective amount” describes a quantity of a specified agent, such as a cyclic peptide or a composition described herein, sufficient to achieve a desired effect in a subject being treated with that agent or composition. For example, this can be the amount of the agent and optionally one or more further therapeutic agents (e.g., one or more further anti-cancer agents), necessary to reduce, alleviate and/or prevent an EGFR-related disease, disorder or condition, such as an EGFR-dependent cancer. Suitably, a “therapeutically effective amount” is sufficient to reduce or eliminate a symptom of an EGFR-related disease, disorder or condition. More particularly, a “therapeutically effective amount” may be an amount sufficient to achieve a desired biological effect, for example an amount that is effective to decrease or prevent disease progression, such as cancer metastasis or recurrence, or overcome resistance to and/or enhance the anti-cancer activity of a further anti-cancer agent.

Ideally, a therapeutically effective amount of an agent is an amount sufficient to induce the desired result without causing a substantial cytotoxic effect in the subject. The therapeutically effective amount of an agent useful for reducing, alleviating and/or preventing the diseases, disorders and conditions described herein will be dependent on the subject being treated, the type and severity of any associated disease, disorder and/or condition (e.g., disease progression), and the manner of administration of the therapeutic composition.

As used herein, the term “subject” refers to any animal, for example, a mammalian animal, including, but not limited to humans, non-human primates, livestock (e.g., sheep, horses, cattle, pigs, donkeys), companion animals (e.g., pets such as dogs and cats), laboratory test animals (e.g., mice, rabbits, rats, guinea pigs), performance animals (e.g., racehorses, camels, greyhounds) or captive wild animals. In various examples, the “subject” is a human. Typically, the terms “subject” and “patient” are used interchangeably, particularly in reference to a human subject. In particular examples, the subject is a male human. In other examples, the subject is a female human.

The terms “EGFR-dependent disease, disorder or condition”, “EGFR-related disease, disorder or condition”, “EGFR-associated disease, disorder or condition” and “disease, disorder or condition associated with EGFR signalling” are used interchangeably herein and refer to disease states and/or symptoms associated with a disease state, where increased levels of EGFR expression and/or activity and/or increased activation of cellular cascades or downstream signalling pathways involving EGFR are found. These terms are also intended to encompass disease states and/or symptoms associated with the activation of alternative (e.g., non-canonical) EGFR signalling pathways. In general, these terms refer to any disorder, the onset, progression or the persistence of the symptoms of which requires the participation of EGFR. Exemplary EGFR-dependent diseases, disorders or conditions include, but are not limited to, for example, cancer, psoriasis (see, e.g., Wang et al., Am J Transl Res, 2019 Feb. 15; 11(2):520-528; Brooks, The Oncologist, 2013; 18:e3-e5) and arthritis (e.g., rheumatoid arthritis; see, e.g., Swanson et al., J Immunol. 2012 Apr. 1; 188(7): 3513-3521).

Suitably, the present methods further including the earlier or initial step of identifying whether the subject's disease, disorder or condition (e.g., a cancer), such as from a sample (e.g., a biopsy sample or a biological sample) obtained from the subject (e.g., from the subject's cancer), is an EGFR-related disease, disorder or condition (e.g., an EGFR-dependent cancer), such as by those methods described herein. In such instances, the present method may include the step of determining an EGFR status, such as an EGFR mutation status or an EGFR expression status, of the subject's disease, disorder or condition, and more particularly, cancer. Well known assays, such as those described herein, can be used for determining the EGFR status of the subject to be treated if such prior determination is desired.

According to alternative examples, the determination or confirmation of the EGFR status of the subject prior to initiating treatment with the cyclic peptide or composition of the present disclosure is not necessary. To this end, there are EGFR-related diseases, disorders or conditions that are commonly known to be EGFR positive such as squamous-cell carcinoma of the head and neck (SCCHN) and colorectal cancer. For such diseases which usually are EGFR positive, the EGFR status of the subject is often not determined prior to treatment with an anti-EGFR agent in prior art approaches. Likewise, in such cases wherein a disease is to be treated wherein, for example, more than 75%, more than 80%, more than 85% or more than 90% of the patients are EGFR positive, treatment with the cyclic peptide or composition of the present disclosure can be initiated without prior determination or confirmation of the EGFR status of the individual subject to be treated.

In view of the above, and according to certain examples, the EGFR-dependent disease, disorder or condition is a cancer. Suitably, the cancer described herein is at least partly mediated by or associated with EGFR or EGFR signalling.

As such, in a particular form, the present disclosure provides a method of treating, preventing or ameliorating an EGFR-related cancer in a subject, said method including the step of administering a therapeutically effective amount of the cyclic peptide or the pharmaceutical composition described herein to the subject to thereby treat, prevent or ameliorate the EGFR-related cancer. In some examples, the subject's cancer is at least partly resistant or refractory to anti-EGFR antibody therapy and/or small molecule EGFR inhibitors, such as those described herein. More particularly, the subject's cancer is at least partly resistant or refractory to cetuximab.

As generally used herein, the terms “cancer”, “tumour”, “malignant” and “malignancy” refer to diseases or conditions, or to cells or tissues associated with the diseases or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumour markers, loss of tumour suppressor expression or activity and/or aberrant or abnormal cell surface marker expression.

Cancers may include any aggressive or potentially aggressive cancers, tumours or other malignancies such as listed in the NCI Cancer Index at http://www.cancer.gov/cancertopics/alphalist, including all major cancer forms such as sarcomas, carcinomas, lymphomas, leukaemias and blastomas, although without limitation thereto. These may include breast cancer, lung cancer inclusive of lung adenocarcinoma, cancers of the reproductive system inclusive of ovarian cancer, cervical cancer, uterine cancer and prostate cancer, cancers of the brain and nervous system, head and neck cancers, gastrointestinal cancers inclusive of colon cancer, colorectal cancer and gastric cancer, liver cancer, kidney cancer, skin cancers such as melanoma and skin carcinomas, blood cell cancers inclusive of lymphoid cancers and myelomonocytic cancers, cancers of the endocrine system such as pancreatic cancer and pituitary cancers, musculoskeletal cancers inclusive of bone and soft tissue cancers, although without limitation thereto.

Exemplary EGFR-related cancers include prostate cancer, non-small cell lung cancer, pancreatic cancer, ovarian cancer, gastrointestinal cancer, rectal cancer, kidney cancer, liver cancer, gallbladder cancer, head and neck cancer, transitional cell carcinoma, squamous cell carcinoma, melanoma, glioblastoma, gliosarcoma, colorectal cancer, breast cancer, oesophageal cancer, bladder cancer, hepatocellular carcinoma, renal cell carcinoma, brain and central nervous system cancer, neuroendocrine cancer, lymphoma, multiple myeloma, and chronic lymphocytic leukaemia. In particular examples, the EGFR-related cancer is prostate cancer.

Suitably, the methods described herein further include the step of administering a further therapeutic agent, such as a further anti-cancer agent, to the subject (i.e., in addition to the agent). As such, the cyclic peptide described herein may be administered alone (i.e., monotherapy) or alternatively be administered in combination with the further therapeutic agent which aims to treat, ameliorate or prevent an EGFR-related cancer or a disease, disorder or condition associated therewith.

In certain examples, the agent described herein may be co-administered with or formulated to be co-administered with (separately, simultaneously or sequentially) a further anti-cancer agent for the treatment of the EGFR-related cancer. Additional cancer treatments (i.e., cancer treatments in addition to the cyclic peptide provided herein) for use in the methods described herein may include drug therapy, chemotherapy, antibody, nucleic acid and other biomolecular therapies, radiation therapy, surgery, nutritional therapy, relaxation or meditational therapy and other natural or holistic therapies, although without limitation thereto. Generally, drugs, biomolecules (e.g., antibodies, inhibitory nucleic acids such as siRNA) or chemotherapeutic agents are referred to herein as “anti-cancer therapeutic agents” or “anti-cancer agents”.

Suitably, the further anti-cancer agent is or comprises one or more of a chemotherapeutic agent, a radiation therapy, a molecularly targeted therapeutic agent and an immunotherapeutic agent.

According to various examples, the further anti-cancer agent is a HER inhibitor, such as an EGFR inhibitor, a HER2 inhibitor, a HER3 inhibitor, a HER4 inhibitor and any combination thereof. As used herein, the term “HER” has its general meaning in the art and refers to a receptor protein tyrosine kinase which belongs to the HER receptor family and includes EGFR, HER2, HER3 and HER4 receptors. As used herein, the term “HER inhibitor” refers to an agent which interferes with HER activation, expression and/or function. Examples of HER inhibitors include HER antibodies (e.g. EGFR, HER2, HER3, or HER4 antibodies); small molecule HER antagonists; HER tyrosine kinase inhibitors; HER2 and EGFR dual tyrosine kinase inhibitors; antisense molecules (see, for example, WO2004/87207); and/or agents that bind to, or interfere with function of, downstream signalling molecules or pathways, such as MAPK or Akt.

In particular examples, the further anti-cancer agent is an EGFR inhibitor. As used herein, the term “EGFR inhibitor” refers to compounds that bind to or otherwise interact directly with EGFR (or any of its sequence variants, deletion or insertion mutants as are known in the art) and prevent or reduce its signalling activity, and is alternatively referred to as an “EGFR antagonist”. Examples of such agents include antibodies and small molecules that bind to EGFR.

Exemplary EGFR inhibitors include the anti-EGFR antibodies: cetuximab (Erbitux®), panitumumab (Vectibix®), matuzumab, nimotuzumab; and small molecule EGFR inhibitors: Tarceva® (erlotinib), IRESSA (gefitinib), osimertinib, EKB-569 (pelitinib, irreversible EGFR TKI), pan-ErbB and other receptor tyrosine kinase inhibitors, lapatinib (EGFR and HER2 inhibitor), pelitinib (EGFR and HER2 inhibitor), vandetanib (ZD6474, ZACTIMA™, EGFR, VEGFR2 and RET TKI), PF00299804 (dacomitinib, irreversible pan-ErbB TKI), CI-1033 (irreversible pan-erbB TKI), afatinib (BIBW2992, irreversible pan-ErbB TKI), AV-412 (dual EGFR and ErbB2 inhibitor), EXEL-7647 (EGFR, ErbB2, GEVGR and EphB4 inhibitor), CO-1686 (irreversible mutant-selective EGFR TKI), AZD9291 (irreversible mutant-selective EGFR TKI), HKI-272 (neratinib, irreversible EGFR/ErbB2 inhibitor) and TAS-121 (mutant-selective EGFR TKI). In particular examples, the further anti-cancer agent is cetuximab, or a variant or derivative thereof.

To this end, it is contemplated that the cyclic peptides of the present disclosure may demonstrate therapeutic benefit when administered as a combination therapy with such EGFR inhibitors. For instance, the ability of the cyclic peptides described herein to reduce EGFR expression levels may overcome what is a known mechanism of acquired resistance to EGFR inhibitors, such as cetuximab (Wheeler et al., 2010, Nature reviews Clinical oncology, 7(9), pp. 493-507) and tyrosine kinase inhibitors (TKIs), such as the third generation EGFR inhibitor TAS-121 (Watanabe et al., 2021, Thoracic cancer, 12(5), pp. 631-642). It is hypothesized that increases in EGFR expression by cancer cells can reduce the effectiveness of EGFR inhibitors, thereby rendering them insufficient to appropriately inhibit EGFR signalling (Nukuga et al., 2017, Cancer research, 77(8), pp. 2078-2089). The cyclic peptides described herein may also show synergy with HER2 and HER3 inhibitors, as these receptors have been linked to gefitinib resistance by providing a mechanism for continued PI3K/Akt pathway activation (Erjala et al. 2006, Wheeler et al. 2010, Nature reviews Clinical oncology, 7(9), pp. 493-507). Further, the cyclic peptides of the present disclosure may show potential synergy with pertuzumab, which targets HER2 heterodimerization (Erjala et al. 2006), as the cyclic peptides may also facilitate the lysosomal degradation of HER2 and HER3 by virtue of their heterodimerization with EGFR.

Referring to other examples, the further anti-cancer agent is a clathrin mediated endocytosis inhibitor. A “clathrin-mediated endocytosis inhibitor” includes a compound or molecule that is capable of specifically inhibiting clathrin-mediated endocytosis, without substantially inhibiting other forms of endocytosis or endocytosis in general. A clathrin-mediated endocytosis inhibitor may include, for example, myr-dyn, chloroquine, ES9, ES9-17, remdesivir, methyl-o-cyclodextrin, rottlerin, and Bis-T. In particular examples, the further anti-cancer agent is chloroquine, or a variant or derivative thereof.

According to some examples, the further anti-cancer agent is an inhibitor of the Ras-Raf-MEK-MAPK pathway. More particularly, the further anti-cancer agent is a MEK (i.e., mitogen-activated protein kinase kinase) inhibitor. The term “MEK inhibitor” is defined herein as a compound or molecule which targets, decreases or inhibits a kinase activity of MEK. Exemplary MEK inhibitors that may be co-administered include U0126, binimetinib, cobimetinib, PD-0325901, pimasertib, RG-7304, trametinib and selumetinib.

It is further contemplated that owing to the structural similarity of the cyclic peptides disclosed herein to the hGIIA-inhibitor c2 and the emerging role of EGFR in inflammation and auto-immune disease (see, e.g., Rayego-Mateos et al., Mediators Inflamm. 2018) that said cyclic peptides may also be effective in preventing and/or treating inflammatory diseases, disorders or conditions, such as psoriasis and rheumatoid arthritis.

Accordingly, in another broad form, the present disclosure provides a method of treating, preventing or ameliorating an inflammatory disease, disorder or condition in a subject, said method including the step of administering a therapeutically effective amount of the cyclic peptide or the pharmaceutical composition described herein to the subject to thereby treat, prevent or ameliorate the inflammatory disease, disorder or condition.

In a related form, the present disclosure provides for the use of a cyclic peptide or a pharmaceutical composition described herein in the manufacture of a medicament for treating, preventing or ameliorating an inflammatory disease, disorder or condition.

As used herein, the term “inflammatory disease, disorder or condition” refers to a large group of diseases, disorders or conditions characterized by abnormal or excessive inflammation, such as that at least partly mediated by EGFR and/or hGIIA (e.g., a non-catalytic activity of hGIIA). The immune system is frequently involved in inflammatory diseases, disorders or conditions, with many immune system disorders resulting in inflammation. In certain examples, the inflammatory disease, disorder or condition is an inflammatory joint disease, disorder or condition, and more particularly arthritis, such as rheumatoid arthritis, osteoarthritis, infectious arthritis, psoriatic arthritis, gouty arthritis and lupus-related arthritis. In other examples, the inflammatory disease, disorder or condition is an inflammatory skin disease, disorder or condition, such as psoriasis.

Suitably, the inflammatory disease, disorder or condition is at least partly mediated or associated with EGFR and/or hGIIA (e.g., an elevated activity and/or signalling thereof).

It is envisaged that the current methods can also improve the prognosis of the subject being treated. For example, administration of a cyclic peptide and/or composition described herein to the subject with an EGFR-related disease, disorder or condition may reduce the probability of a clinical worsening event (e.g., metastasis, cancer progression, cancer recurrence or a combination thereof) during the treatment period, and/or a modulation (i.e., an increase or decrease) from baseline in one or more biomarkers of disease progression. In some examples, the methods described herein provide a reduction of at least about 25%, at least about 50%, at least about 75% or at least about 80%, in probability of a clinical worsening event during the treatment period. In other examples, the current methods prevent a change of at least about 25%, at least about 50%, at least about 75% or at least about 80% of the concentration of one or more biomarkers of EGFR-related disease, disorder or condition progression.

### Inhibiting EGFR in a Cell

The present disclosure also provides methods of inhibiting EGFR in a cell. In this regard, the present inventors have surprisingly shown that the cyclic peptides described herein can promote internalisation, sequestration and lysosomal degradation of EGFR in cells. As a result, the cyclic peptides disclosed herein can be selectively toxic to EGFR-dependent cancer cells and thereby inhibit their viability and/or growth.

Accordingly, in one form, the present disclosure provides a method of inhibiting EGFR, such as EGFR signalling, activity or expression, in a cell, said method including the step of contacting the cell with an effective amount of the cyclic peptide or the pharmaceutical composition described herein to thereby inhibit EGFR in the cell.

Suitably, a level of EGFR signalling, activity or expression in the cell in the presence of the cyclic peptide is less than about 99%, 98%, 97%, 96%, 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level of EGFR signalling, activity or expression of a control or reference cell in the absence of the cyclic peptide. Such levels of EGFR signalling, activity and expression may be determined by any means known in the art, such as those described herein.

In a related form, the present disclosure provides a method of promoting lysosomal degradation of EGFR in a cell, said method including the step of contacting the cell with an effective amount of the cyclic peptide or the pharmaceutical composition provided herein to thereby promote lysosomal degradation of EGFR in the cell.

Lysosomal degradation refers to the degradation of unwanted materials, such as macromolecules, foreign material, defective or unwanted proteins or other cellular organelles, by lysosomes in a cell. Lysosomes are those membrane-bound lytic organelles that contain hydrolases active at acid pH within cells. Lysosomal degradation may be assessed by any means known in the art, such as a reporter assay (e.g., the RFP/GFP reporter assay of Tomihari et al., STAR Protoc, 2021), EGFR expression levels, or confocal microscopy to determine the cellular localisation of EGFR, such as in Example 1 using LysoTracker.

Suitably, a level of lysosomal degradation of EGFR in the cell in the presence of the cyclic peptide is more than about 101%, 102%, 103%, 104%, 105%, 110%, 120%, 130%, 140%, 150%, 160%, 170%, 180% or 190%, or even more than about 200%, 250%, 300%, 350%, 400%, 450% or 500% of the level of lysosomal degradation of EGFR in a control or reference cell in the absence of the cyclic peptide. Such levels of lysosomal degradation may be determined by any means known in the art, such as those described herein.

In another form, the present disclosure relates to a method of reducing the viability and/or growth of a cancer cell, said method including the step of contacting the cancer cell with an effective amount of the cyclic peptide or the pharmaceutical composition disclosed herein to thereby reduce the viability and/or growth of the cancer cell.

The term “reducing the viability and/or growth” is used to mean reducing the ability of a cancer cell, such as an EGFR-dependent cancer cell, to survive and/or proliferate. Cell viability and/or growth may be inhibited in any measurable amount. Inhibition of cell viability and/or growth may be complete or may be partial. Thus, the methods disclosed herein may comprise at least partial inhibition of cancer cell viability and/or growth.

Suitably, the viability and/or growth of the cancer cell in the presence of the cyclic peptide is less than about 99%, 98%, 97%, 96%, 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the viability and/or growth of a control or reference cancer cell in the absence of the cyclic peptide.

Viability of the cancer cells may be assessed by any suitable means or method known in the art. Typically, cell viability is quantified as the percentage of living cells present (i.e., the number of live cells divided by the total number of cells). As cells are either live or dead, cell viability may be determined by quantifying either dead cells or live cells. One suitable method is trypan blue exclusion. Other assays for determining cell viability may include, without limitation, propidium iodide staining, TUNEL, Resazurin, methyl violet, lactate dehydrogenase, fluorescein diacetate hydrolysis, MTT, caspase, and ATP assays.

Growth or proliferation of the cancer cells may also be measured by methods known in the art. Suitable methods include measuring the size of the cell population (e.g., by counting cells using a marker specific for the cell population, i.e. a tumour specific marker) or by performing cell cycle analysis using 5-bromo-2′-deoxyuridine (BrdU) which becomes incorporated into newly made DNA and propidium iodide (PI) and analysing by flow cytometry in combination with a cell population specific marker. Other suitable methods for measuring proliferation include proliferations assays, such as MTT assays, MTS assays, intracellular ATP assays, measuring cellular LDH activity, 3H-thymidine incorporation and the Cell Titer Glo Assay.

In relation to the aforementioned methods, the cell is suitably a cancer cell and more particularly an EGFR-related cancer cell. The EGFR-related cancer cell may be derived from any vertebrate, such as a mammal, and in particular, a human. Additionally, the EGFR-related cancer cell may be derived from any cancer, such as those described herein. In some examples, the cancer cell is a prostate cancer cell, a non-small cell lung cancer cell, a pancreatic cancer cell, an ovarian cancer cell, a gastrointestinal cancer cell, a rectal cancer cell, a kidney cancer cell, a liver cancer cell, a gallbladder cancer cell, a head and neck cancer cell, a transitional cell carcinoma cell, a squamous cell carcinoma cell, a melanoma cell, a glioblastoma cell, a gliosarcoma cell, a colorectal cancer cell, a breast cancer cell, an oesophageal cancer cell, a bladder cancer cell, a hepatocellular carcinoma cell, a renal cell carcinoma cell, a brain and central nervous system cancer cell, a neuroendocrine cancer cell, a lymphoma cell, a multiple myeloma cell, or a chronic lymphocytic leukaemia cell. According to particular examples, the cancer cell is a prostate cancer cell.

As used herein, the term “effective amount” and the like refers to an amount of the cyclic peptide or pharmaceutical composition that is sufficient to induce a desired physiological outcome (e.g., at least partly inhibiting EGFR in the cell, at least partly promoting lysosomal degradation of EGFR in the cell, at least partly reducing the viability and/or growth of the cancer cell). An effective amount can be administered in one or more administrations, applications or dosages.

It is envisaged that the method of the present methods may be performed in relation to cells in vitro, in vivo and/or ex vivo. In some examples, the method is performed in vitro, such as with EGFR-dependent cancer cells isolated from a subject or a patient-derived xenograft model. Accordingly, in some examples, the present method includes the earlier step of isolating the cells expressing EGFR, such as EGFR-dependent cancer cells, from the subject. In other examples, the present method is performed in vivo in a subject.

For the purposes of the present disclosure, by “isolated” is meant material that has been removed from its natural state or otherwise been subjected to human manipulation. Isolated material (e.g., EGFR-dependent cancer cells) may be substantially or essentially free from components that normally accompany it in its natural state, or may be manipulated so as to be in an artificial state together with components that normally accompany it in its natural state.

The present method may include the initial or earlier step of determining an EGFR status of a cell, such as an EGFR mutational or expression status of a cancer cell to identify an EGFR-dependent cancer cell. Such determining, assessing, evaluating or assaying an EGFR status of a cancer cell, may be performed by any technique known in the art, such as those methods described herein.

According to certain examples, the present methods further include the step of contacting the EGFR-related cancer cell with an effective amount of a further anti-cancer agent, such as those described herein.

### Methods of Screening

In light of the present disclosure, the person skilled in the art may conduct screening assays to identify, design or produce an agent, such as a cyclic peptide, a peptidomimetic or a small molecule, capable of interacting with or binding to a ligand binding domain of EGFR.

In a particular form, the present method includes the steps of:
(a) contacting a cell expressing EGFR with a candidate agent, wherein the candidate agent is a derivative, variant or analogue of: (i) cyclo-((2-Nal)-Leu-Ser-(2-Nal)-Arg); or (ii) the cyclic peptide described herein; and
(b) determining whether the candidate agent inhibits the binding of an EGFR ligand to EGFR of the cell.

In some examples, the present method further includes the step of contacting the cell with the EGFR ligand, such as before, after or simultaneously with the candidate agent.

In a related form, the present disclosure relates to an agent capable of interacting with or binding to a ligand binding domain of EGFR, obtained by the method described herein.

The EGFR ligand may be any known in the art, such as those described herein. In particular examples, the EGFR ligand is EGF. In other examples, the EGFR ligand is hGIIA.

According to various examples, the cell expressing EGFR is a cancer cell, such as those described herein. In certain examples, the cell expressing EGFR is an EGFR-related cancer cell.

Suitably, the ligand binding domain of EGFR comprises or is defined by one or more residues selected from amino acids Q8, G9, T10, S11, N12, K13, L14, T15, Q16, L17, H23, F24, L25, S26, L27, Q28, R29, M30, L41, E42, 143 and T44 of SEQ ID NO: 2. More particularly, the ligand binding domain of EGFR suitably comprises or is defined by one or more residues selected from amino acids L17, H23, F24, L25, S26, L27, Q28, R29, M30, L41, E42, 143 and T44 of SEQ ID NO: 2.

It is envisaged that any method known in the art to assess EGFR ligand binding and/or EGFR activity, such as those described herein, may be utilised for the present method. In particular examples, a level of EGFR expression and/or activity, such as by way of a kinase assay, is assessed. In other examples, a level of EGFR ligand binding to EGFR, such as by way of an immunoassay (e.g., ELISA, AlphaLISA), is assessed.

The effect of the presence of the candidate agents on lysosomal degradation of EGFR may also be assessed. This may be achieved, for example, through determining the level of EGFR expression and/or activity and/or determining a cellular or subcellular localisation of EGFR in lysosomes (e.g., confocal microscopy and lysotracker).

In another example, the effect of the presence of the candidate agents on the viability and/or growth of a cell, such as a cancer cell, may also be assessed. This may be achieved, for example, by those methods hereinbefore described.

Suitably, the candidate agent is a cyclic peptide. It is also envisaged, however, that the candidate agent may comprise, for example, a linear peptide, a peptidomimetic or a small molecule that utilise the binding structures or moieties of the cyclic peptides of the present disclosure in an attempt to mimic their binding to a ligand binding domain of EGFR. The cyclic peptides described herein may also be utilised to search existing compound libraries, numbering hundreds of thousands to millions of candidate inhibitors (chemical compounds including synthetic, small organic molecules or natural products, such as inhibitory peptides or proteins), which may be screened or tested for biological activity at any one of hundreds of molecular targets in order to find potential new drugs, or lead compounds. Screening methods may include, but are not limited to, computer-based (“in silico”) screening and high throughput screening based on in vitro assays.

Thus, the present disclosure provides methods to identify, design or produce an agent capable of interacting with or binding to a ligand binding domain of EGFR when administered to a cell, tissue, or subject. Such methods may be carried out in vivo, for example in animal subjects; or using in vitro and/or ex vivo assays, such as described herein.

Typically, the active compounds, or “hits”, from this initial screening process are then tested sequentially through a series of other in vitro and/or in vivo tests to further characterize the active compounds. A progressively smaller number of the “successful” compounds at each stage are selected for subsequent testing, eventually leading to one or more drug candidates being selected to proceed to being tested in human clinical trials.

At the clinical level, screening a candidate agent may include obtaining samples from test subjects before and after the subjects have been exposed to a test compound. The levels in the samples of EGFR expression, activity or signalling may then be measured and analysed to determine whether the levels and/or activity thereof changes after exposure to a candidate agent. By way of example, protein product levels in the samples may be determined by mass spectrometry, western blot, ELISA, electrochemistry and/or by any other appropriate means known to one of skill in the art.

In this regard, candidate agents that are identified of being capable of reducing, eliminating, suppressing or inhibiting the expression level and/or activity of EGFR may then be administered to patients who are suffering from an EGFR-dependent cancer. For example, the administration of a candidate agent which inhibits or decreases the activity and/or expression of EGFR may treat the EGFR-dependent cancer and/or decrease the risk or progression of the EGFR-dependent cancer, if the increased activity and/or expression of EGFR is responsible, at least in part, for the progression and/or onset of said EGFR-dependent cancer.

In various examples, the methods disclosed herein may further include the step of isolating and/or purifying an agent that inhibits the binding of the EGFR ligand to EGFR.

In particular examples, the method further includes the step of formulating the isolated and/or purified agent into a pharmaceutically acceptable formulation. Other steps in the method may include sterilising the formulation and/or filling the formulation into a container, such as a vial, an ampoule, a bag, a blister pack, a bottle, a cartridge, an injection needle, an injection syringe, a single dose container, a strip of multiple single dose containers, or a tube.

It is also contemplated that the candidate agent or inhibitor may be rationally designed or engineered de novo based on desired or predicted structural characteristics or features that indicate the candidate agent could block or inhibit binding of an EGFR ligand to EGFR. In other examples, the candidate agent may be identified by screening a library of molecules without initial selection based on desired or predicted structural characteristics or features that indicate the candidate modulator could block or inhibit binding of an EGFR ligand to EGFR. Such libraries may comprise randomly generated or directed libraries of proteins or peptides, and more particularly cyclic peptides, libraries of naturally-occurring molecules and/or combinatorial libraries of synthetic organic molecules.

Non-limiting examples of techniques applicable to the design and/or screening of candidate agents may employ X-ray crystallography, NMR spectroscopy, computer assisted screening of structural databases, computer-assisted modelling or biochemical or biophysical techniques which detect molecular binding interactions, as are well known in the art.

Biophysical and biochemical techniques which identify molecular interactions include competitive radioligand binding assays, co-immunoprecipitation, fluorescence-based assays including fluorescence resonance energy transfer (FRET) binding assays, electrophysiology, analytical ultracentrifugation, label transfer, chemical cross-linking, mass spectroscopy, microcalorimetry, surface plasmon resonance and optical biosensor-based methods, such as provided in Chapter 20 of CURRENT PROTOCOLS IN PROTEIN SCIENCE Eds. Coligan et al., (John Wiley & Sons, 1997) Biochemical techniques such as two-hybrid and phage display screening methods are provided in Chapter 1 of CURRENT PROTOCOLS IN PROTEIN SCIENCE Eds. Coligan et al, (John Wiley & Sons, 1997).

Accordingly, an earlier step of the method may include identifying a plurality of candidate agents that are selected according to broad structural and/or functional attributes, such as an ability to bind a ligand binding domain of EGFR.

Additionally, the present method may further include one or more of the steps of: selecting a candidate agent that inhibits or blocks binding of the EGFR ligand to EGFR of the cell;
selecting a candidate agent that promotes lysosomal degradation of EGFR in the cell;
selecting a candidate agent that reduces the growth and/or viability of the cell;
isolating or purifying the candidate agent;
formulating the candidate agent into a pharmaceutical formulation; and
adding the candidate agent or the pharmaceutical formulation to packaging and/or a container, such as a vial, ampoule, bag, blister pack, bottle, cartridge, injection needle, injection syringe, single dose container, strip of multiple single dose containers, or tube.

### Methods of Production

In general, the cyclic peptides disclosed herein, or of a formula disclosed herein, may be synthesized by any means known in the art, including by solid-phase synthesis, and may be purified according to methods known in the art. Any of a number of well-known procedures utilizing a variety of resins and reagents may be used to prepare the peptides disclosed herein, or of a formula disclosed herein.

Solid phase peptide synthesis methods are well known and practiced in the art. In such methods the synthesis of peptides of the invention can be carried out by sequentially incorporating the desired amino acid residues one at a time into the growing peptide chain according to the general principles of solid phase methods.

The cyclic peptides disclosed herein, or of a formula disclosed herein, may be readily synthesized by known conventional procedures for the formation of a peptide linkage between amino acids. Such conventional procedures include, for example, any solution phase procedure permitting a condensation between the free alpha amino group of an amino acid residue having its carboxyl group and other reactive groups protected and the free primary carboxyl group of another amino acid residue having its amino group or other reactive groups protected. Suitably, the cyclic peptides disclosed herein, or of a formula disclosed herein, may be synthesized by solid-phase synthesis and purified according to methods known in the art. Any of a number of well-known procedures utilizing a variety of resins and reagents may be used to prepare the peptides disclosed herein, or of a formula disclosed herein.

The process for synthesizing the cyclic peptides may be carried out by a procedure whereby each amino acid residue in the desired sequence is added one at a time in succession to another amino acid residue or by a procedure whereby peptide fragments with the desired amino acid sequence are first synthesized conventionally. The resulting linear peptide is then cyclized in solution phase to yield a cyclic peptide of the present disclosure.

### EXAMPLES

### Example 1. Lysosome Targeting Peptides (LYTAPS) that Bind the Extracellular Domain of EGFR and Initiate its Lysosomal Degradation

In this Example, the inventors sought to design a new class of EGFR inhibitor—lysosomal targeting peptides (LYTAPs), that bind to the extracellular domain of EGFR and initiate non-CME and lysosomal degradation.

### Materials & Methods

### Compound Synthesis

Compounds D1-D3 were synthesised by Auspep Ltd (Tullamarine, VIC. Australia) as a 95% purity acetate salt. FLM-c2 was manufactured by Bachem Ltd (Bubendorf, Switzerland) to >95% purity as an acetate salt.

### Molecular Modelling

The crystal structure of EGFR complexed with EGF ligand (PDB 1nql: Ferguson et al, 2003) was used as the target in the modelling study. FLM-C2 analogue Cyclo[(D-Phe)LS(L-Phe)R] was docked to the crystal structure of EGFR using software quCBit (www.medchemsoft.com). The predicted binding structure of FLM-C2 analogue to EGFR indicates that 2-Nal of FLM-C2 is located around a small cavity of EGFR. The inventors posited that a change of 2-Nal to 1-Nal with D chirality will direct the D-1-Nal into the cavity and stabilise the binding of FLM-C2 derivatives to EGFR and more importantly, block EGFR/EGF interactions.

### Cell Viability Assay

To quantify cell toxicity of FLM-c2, the MTS (3-(4,5-dimethylthiazol-2-yl)-5-(3-carboxymethoxyphenyl)-2-(4-sulfophenyl)-2H-tetrazolium) assay was performed using CellTiter 96@AQueous One Solution Cell Proliferation Assay reagent (Promega, Madison, WI), as per the manufacturer's instructions. PC-3 cells were seeded at 2000 cells per well on a 96 well plate overnight at 37° C. and 5% C02. Cells were then treated with 0-250 μM with each of FLM-c2, D1, D2 and D3, which was incubated for 72 hours at 37° C. and 5% C02. Medium was replaced with 100 μL/well of phenol red-free MEM (Sigma-Aldrich) and 20 μL/well of the MTS reagent was added. After incubation for 3 hours in 37° C. with 5% C02, the absorbance was measured at 490 nm with Spectramax M5e.

### EGF-EGFR AlphaLISA Assay

The AlphaLISA EGF/EGFR binding kit (Perkin-Elmer) was used as per the manufacturer's instructions. Briefly, in a 384 AlphaPlate (Perkin-Elmer), 5 μL streptavidin beads were added to each well, along with 5 μL of biotintylated EGF. Next, 5 μL of FLM-c2, D1-D3 or erlotinib were added to each well for a final concentration of 0.01-200 μM. Finally, 5 μL of EGFR acceptor beads were added, and fluorescence imaged on a plate reader.

### Western Blot

PC-3 cells were seeded in a 6 well plate at 200,000 cells/well overnight at 37° C. and 5% C02. Media was removed, cells were washed with PBS and serum free media added 1 hour before treatment with FLM-c2, D1-D3 for 3-48 hours. After treatment, media was removed and cells were washed with ice cold PBS. PBS was removed and cells were scraped into 1 mL of PBS containing 5.3 mM of EDTA and centrifuged at 2500 g for 2 min. PBS was removed and cells were lysed with lysis buffer (PBS, 0.1% NP-40, 0.1% sodium deoxycholate, 150 mM NaCl, 1 mM EDTA, 1 mM dithiothreitol, 1 mM phenylmethylsulfonyl fluoride, and IX protease inhibitor cocktail)¹⁷ and mixed with RM-2L Intelli-mixer for 30 min at 4° C. Lysates were then centrifuged at 12,000 g for 20 min at 4° C. 20 μg of lysate was added to 4 μL of Bolt reducing agent and 10 μL of reducing buffer (both reagents supplied by Thermo Fisher Scientific) for a final volume of 40 μL with PBS. Samples were boiled and run on a gel, transferred to a membrane and blocked for 1 hour in 5% skim milk at 25° C. Membrane was probed with either EGFR antibody #2232, GAPDH antibody #2118 or BS-Actin antibody #4970 (Cell Signalling) at 1:1000 in 5% skim milk/TBST overnight at 4° C. Each membrane was washed three times with 50 mL TBST for 5 mins and then incubated with the relevant secondary α-rabbit IgG-HRP-linked antibody (1:1000), for 1 hour at room temperature. The membrane was washed as described above and imaged with the Licor Odyssey.

### Co-Immunoprecipitation

PC-3 cells were seeded in a 6-well plate at 300,000 cells/well and grown overnight at 37° C. and 5% CO2. Cells were then incubated with 2 μg/mL hGIIA with and without FLM-c2 (100 μM) for two hours. After incubation, cells were washed three times with 10 mL ice cold PBS for 2 min. PBS was removed and cells were scraped into 1 mL of PBS containing 5.3 mM of EDTA and lysed as described above. Lysates were then centrifuged at 12,000 g for 20 min at 4° C. and incubated with 10 lag of α-hGIIA and 10 μL of protein G agarose beads (Abcam, Cambridge, UK) and overnight incubation at 4° C. Bead lysate mixture was centrifuged at 1000 g for 5 min and lysate was removed, with beads washed three times with 1 mL of PBS and subsequent centrifugation at 1000 g for 5 min and removal of supernatant. Beads were run on a gel, transferred to a membrane, blocked, probed with EGFR antibody, and imaged as described above.

### Live Cell Imaging

PC-3 cells were seeded in a 24 well glass-bottom plate at 50 000 cells/well and incubated overnight at 37° C. and 5% CO₂. Complete media was removed, wells washed with 500 μL of warm PBS for 1 min then removed and 500 μL serum free medium was added and left to incubate for one hour at 37° C. and 5% CO2. Cells were co-transfected with plasmids encoding fusion protein EGFR-EGFP (Addgene plasmid #32751, a gift from Alexander Sorkin) using Lipofectamine 3000 (Thermo Fisher Scientific) as per the manufacturer's instructions. Cells were incubated for 48 hours at 37° C. and 5% CO2, after which transfection efficiency was determined through observation of EGFP and signal with a confocal microscope LSM 800 (Zeiss). LysoTracker far red (Thermo Fisher Scientific) was incubated at 1 μM for 37° C. and 5% CO2. Cells were washed three times with PBS and serum free media was added to cells, Either DMSO (0.5%) or D3 (100 μM) were added overnight at 37° C. and 5% CO2 before imaging on the confocal microscope LSM 800 (Zeiss).

### Results

FLM-C2 Binds to EGFR, Blocking the Interaction with EGFR Ligand hGIIA

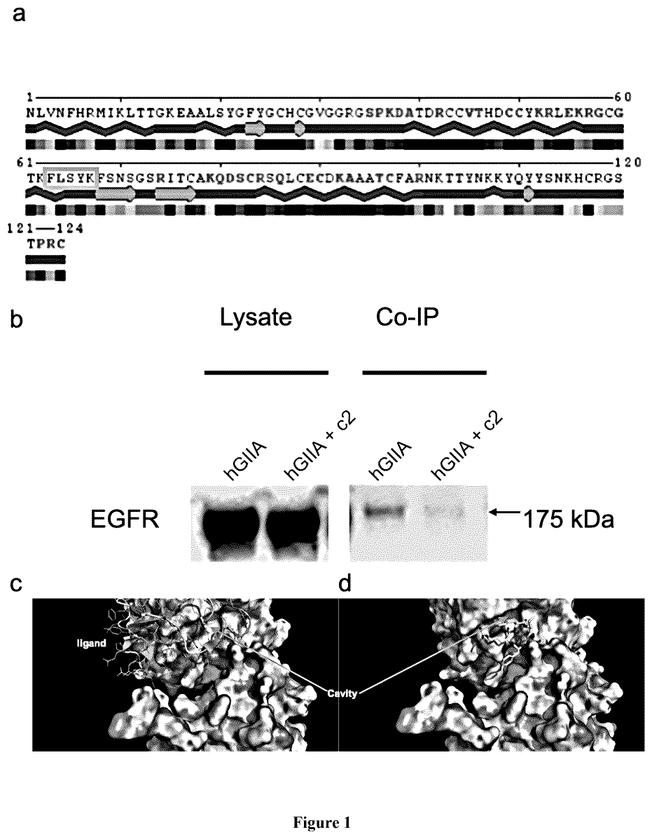

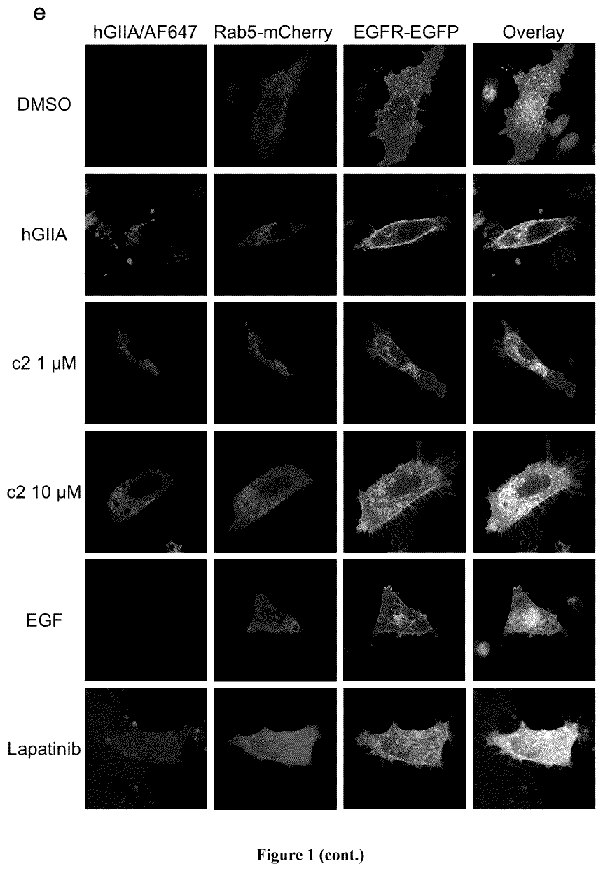

FLM-c2 is a cyclic peptide (cyclo-((2-Nal)-Leu-Ser-(2-Nal)-Arg) derived from the structure of a secreted phospholipase A₂ (sPLA₂) known as human sPLA₂-IIA or human group IIA sPLA₂ (hGIIA)¹⁵. Recently, hGIIA has been identified as a ligand for EGFR and HER-2^{16,17}. We have found that the sequence of FLM-c2, derived from hGIIA (aa70-74), is located between two sequences of hGIIA predicted to bind EGFR¹⁶, suggesting its potential to inhibit this interaction (FIG. 1a). This was confirmed in the prostate cancer cell line PC-3, where an EGFR signal was detected by Western blot following co-immunoprecipitation with hGIIA. FLM-c2 was able to inhibit this interaction (FIG. 1b). Further, molecular modelling also indicated that FLM-c2 binds near the cavity of EGFR's extracellular domain, in the same region as its primary ligand EGF, again indicating its potential to block multiple EGFR ligands (FIGS. 1c and 1d).

The effect of c2 on EGFR, Rab5 and hGIIA localisation was also examined in PC-3 cells. PC-3 cells were co-transfected with EGFR-EGFP and Rab5-mCherry before treatment with hGIIA/AF647 and c2 at 0, 1 and 10 μM. DMSO treated cells exhibit punctate Rab5 and EGFR expression, indicating that some EGFR has already become endocytosed. hGIIA/AF647 treated cells show an increased localisation of EGFR on the surface of the cell, as well as strong colocalisation of hGIIA/AF647 with Rab5-mCherry and some colocalisation with EGFR-GFP (FIG. 1e). Colocalisation between hGIIA, rab5 and EGFR indicates that some hGIIA becomes endocytosed with EGFR after activation. c2 treated cells also exhibit similar localisations, however with c2 treatment at 10 μM PC-3 cells exhibit increased hGIIA/AF647 and Rab5-mCherry punctate size, as well as circularisation of EGFR-GFP expression (FIG. 1e). EGF at 25 ng/mL was used as a positive control for EGFR activation, which resulted in circularisation of Rab5-mCherry. Lapatinib—a clinically used EGFR inhibitor—was used at 5 μM as a control for EGFR inhibition, altering the localisation of hGIIA and Rab5 to become less punctate and more cytosolic (FIG. 1e). Co-incubation with c2 at higher concentrations (10 μM) increases the size of hGIA/AF647 and Rab5-mCherry positive vesicles, as well as the circularisation of EGFR (FIG. 1e), again indicating the endocytotic pathway of EGFR and hGIIA is modulated.

### FLM-c2 and Derivatives Block Key EGFR Ligand EGF

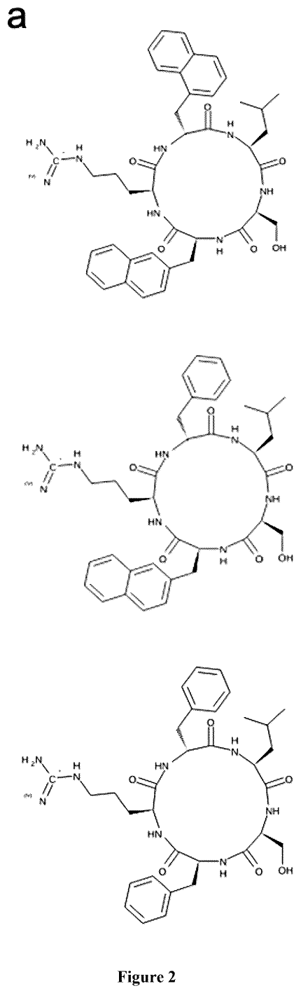

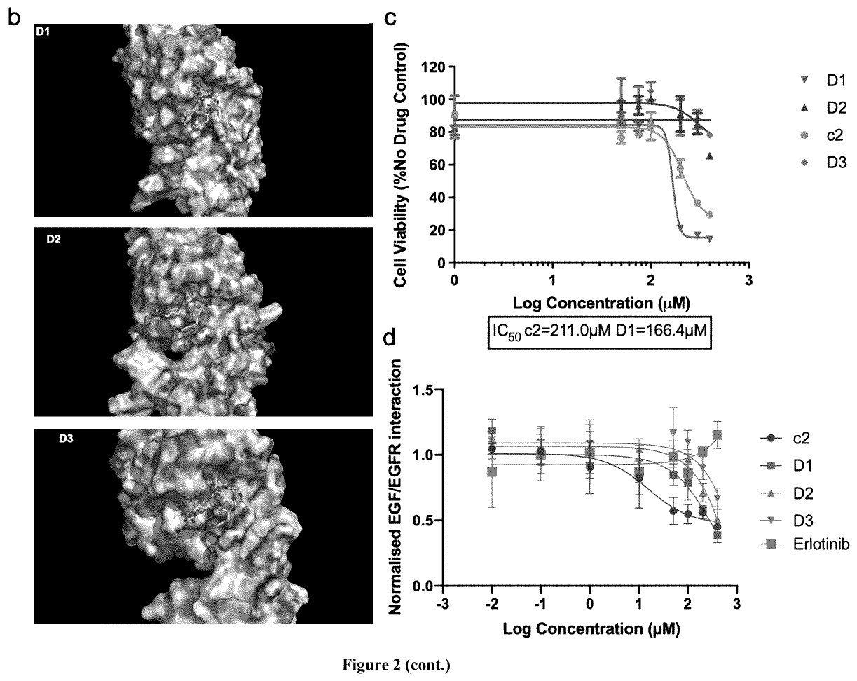

Based on these results, derivatives of FLM-c2 were designed; D1, (cyclo[(D-1Nal)-Leu-Ser-(L-2Nal)-Arg]), D2 (cyclo[(D-Phe)-Leu-Ser-(L-2Nal)-Arg]) and D3, cyclo[(D-Phe)-Leu-Ser-(L-Phe)-Arg)](FIG. 2a). These compounds were designed specifically to bind the cavity of EGFR's extracellular domain where the EGF ligand binds seq: 23-HFLSLQRM-30, 41-LETT-44, L17 (FIG. 2b). The IC₅₀ of these compounds was investigated in the prostate cancer (PCa) cell line PC-3 (FIG. 2c), with D1 being more potent IC₅₀ (166 μM) than FLM-c2 (211 μM). D2 and D3 did not produce an IC₅₀ under 250 μM. The ability of these compounds to inhibit the EGF-EGFR interaction was investigated using an in vitro AlphaLISA assay (Perkin Elmer). FLM-C2 and derivatives all inhibited the EGF/EGFR interaction at high concentrations (potency FLM-c2>D1>D2>D3), whereas the EGFR TKI erlotinib did not (FIG. 2d).

### Derivatives, but not FLM-c2, Cause Lysosomal Degradation of EGFR

The mechanism of action of these derivatives was investigated further in PC-3. Cells were treated with either DMSO (control) FLM-c2 (100 μM) D1-D3 (100 μM) or EGF (50 ng/mL) for 24 hours. Cells were lysed and EGFR was detected by western blot. All derivatives (D1-D3) showed EGFR degradation, whereas FLM-c2 did not, suggesting a different mechanism of action. To confirm EGFR was degraded via lysosomes, PC-3 cells incubated with DMSO or D3 (100 μM) for 24 hours were imaged. Cells were tagged with EGFR-EGFP and lysotracker, which confirmed colocalization of EGFR with lysosomes.

### Time-Course Assay Reveals LYTAP D3 Retains Potency Over 48 Hours

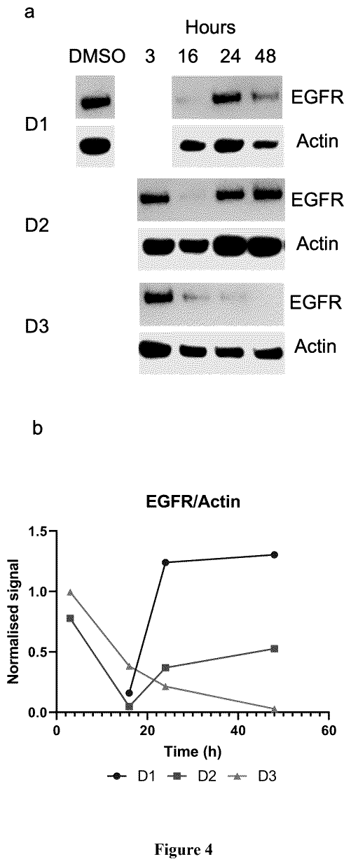

PC-3 cells were treated with D1, D2 and D3 at 100 μM for 3, 16, 24 and 48 hours. EGFR signal was normalised against ß-Actin, and against DMSO control. D1 and D2 initially degraded EGFR by 84% and 95%, respectively, but EGFR levels had returned to normal by 24 and 48 hours (FIGS. 4a and 4b). D3 treatment resulted in a sustained depletion of EGFR, estimated at over 97% after 48 hours (FIGS. 4a and 4b).

### LYTAPs Cause EGFR Internalisation and Lysosomal Degradation, Reducing EGFR Signalling

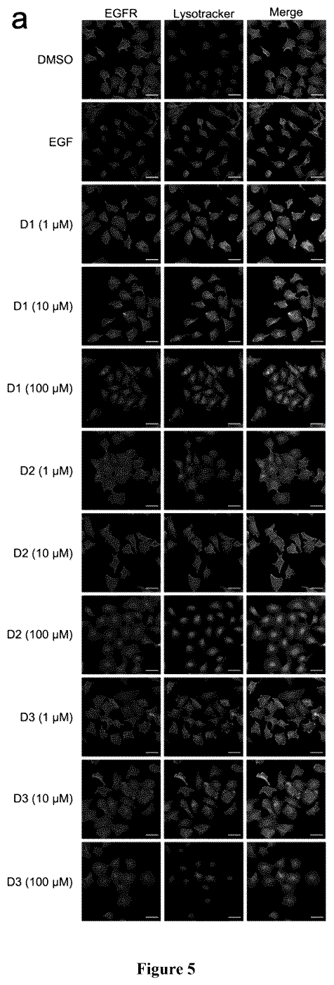

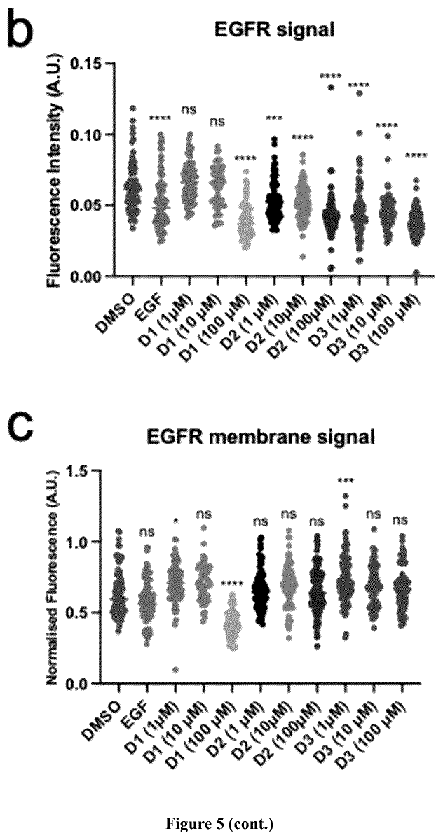

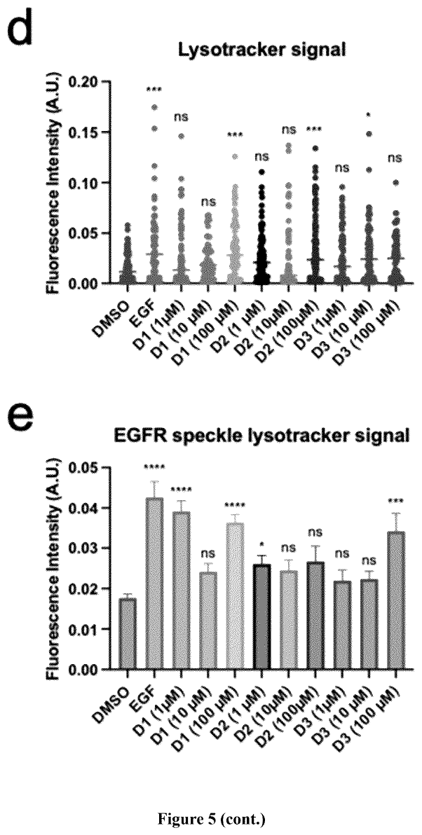

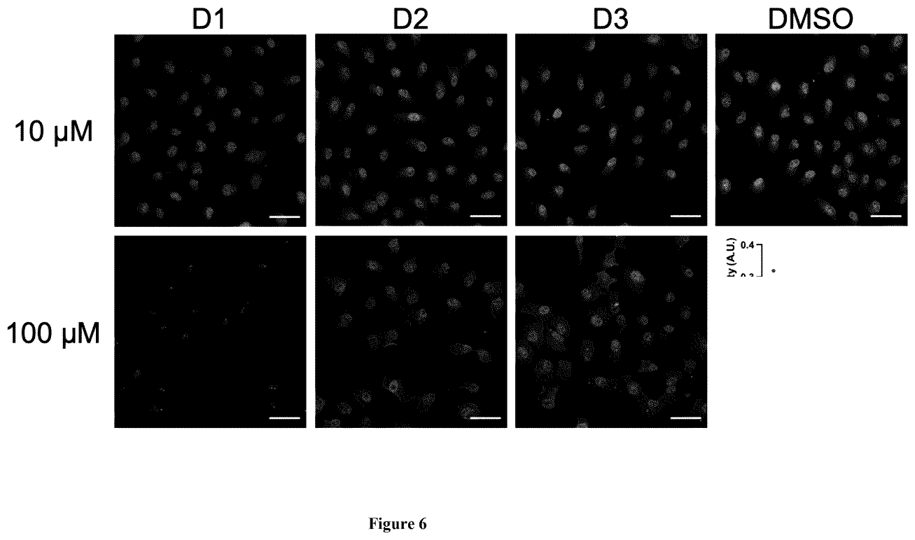

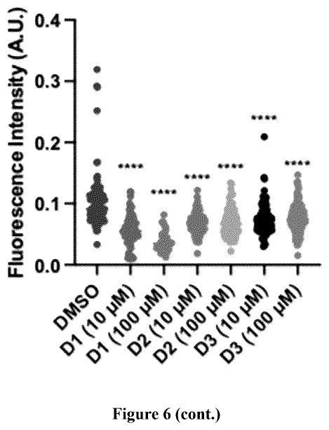

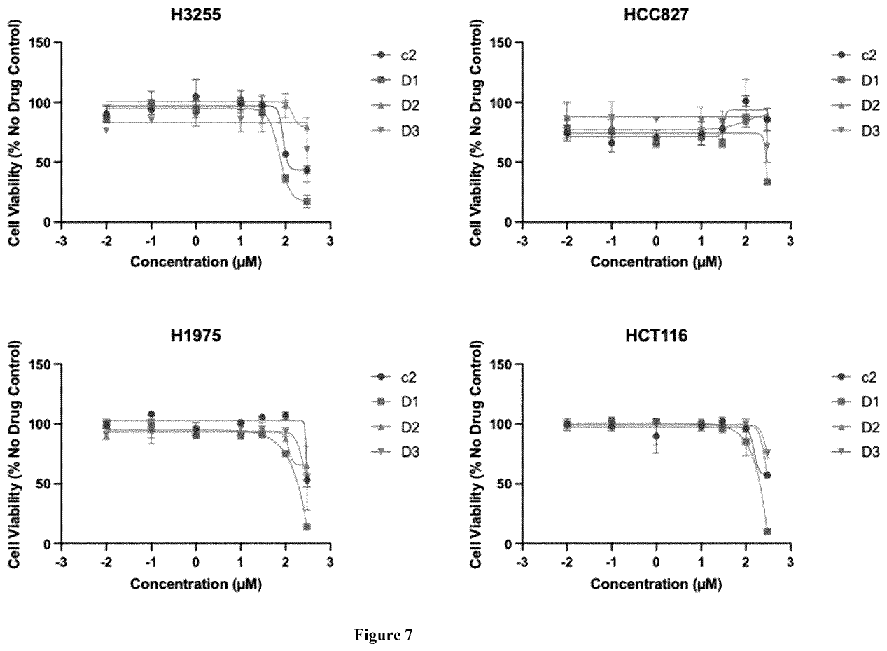

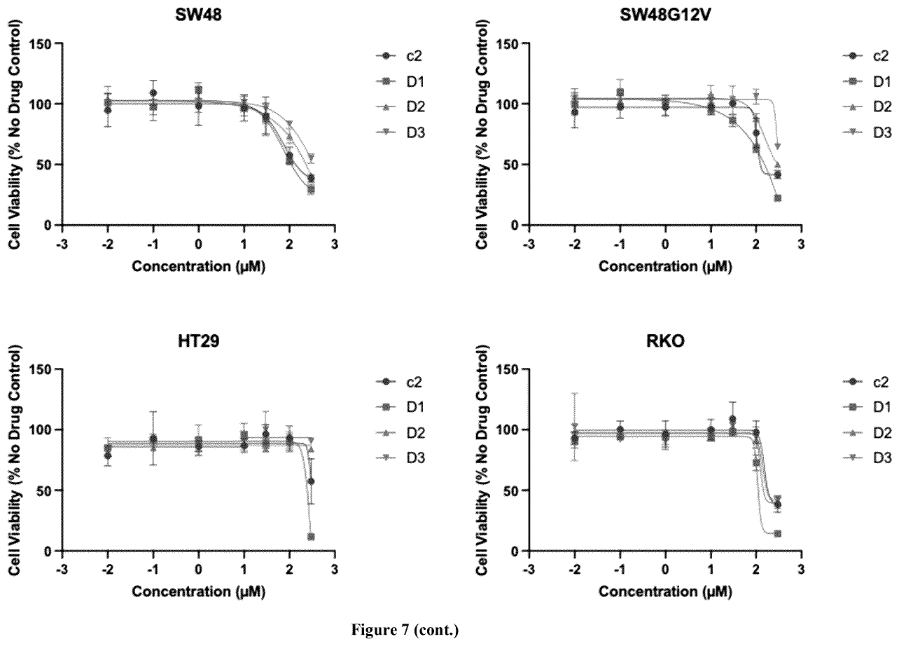

To investigate the mechanism of how LYTAPS cause EGFR degradation, LYTAPs D1-D3 (1-100 μM) were incubated on A549 cells for 16 hours, with DMSO (0.5%) and high EGF (50 ng/mL) used as controls. Cells were then stained with Lysotracker, fixed and stained with EGFR-antibody. Under DMSO control, the majority of EGFR signal is present at the membrane (FIG. 5a). All LYTAPs cause a significant reduction in EGFR signal at concentrations as low as 1 μM (D1 P<0.0001, D2 P=0.0006, D3 P<0.0001), similarly to positive control EGF (P<0.0001, one-way ANOVA) (FIG. 5b). D1 at 100 μM also significantly reduced the fraction of membrane-localised EGFR signal in comparison to DMSO control (P<0.0001), whereas EGF did not (P=0.0925) (FIG. 5c). D1 and D2 at 100 μM also increased lysotracker signal (P=0.0005, and P=0.0005, respectively), similarly to EGF (P=0.0003) (FIG. 5d). Of the endocytosed EGFR ‘speckles’, there is significantly increased lysotracker signal in EGF (P<0.0001), D1 1 μM (P<0.0001)’ and 100 μM (P<0.0001), D2 at 1 μM (P=0.0156) and D3 at 100 μM (P=0.0007), indicating these speckles are lysosomes (FIG. 5e).

To examine whether LYTAPs can inhibit EGFR downstream signalling, A549 cells were pre-incubated with 10-100 μM of D1-D3 for 16 hours, before stimulation with EGF (10 ng/mL) for 10 min. Cells were then fixed and immunofluorescently stained for pERK. All LYTAPs at all both 10 and 100 μM concentrations significantly reduce pERK signal (all P<0.0001, one-way ANOVA), with D1 at 100 μM having the largest effect.

### LYTAP D1 Retains Efficacy in Cell Lines Sensitive and Resistant to Erlotinib and Cetuximab

The therapeutic benefit of LYTAPs was investigated in both CRC and NSCLC cancer cell lines with known sensitivities to clinically used mABs and TKIs (Table 1). Cells were treated with c2, D1-D3 and proliferation assays were conducted, which found D1 had the lowest IC₅₀ in all cell lines. Furthermore, D1 had comparable efficacy in cell lines both cetuximab sensitive (SW48) cetuximab resistant (SW48G12V, HT29, HCT116 and RKO), as well as retained efficacy in NSCLC cell lines sensitive to erlotinib (HCC827 and H1975) and resistant to erlotinib (H3255).

TABLE 1
EGFR/KRAS/BRAF status of CRC and NSCLC cell lines
and resistance to clinical EGFR inhibitors.

| | | EGFR/KRAS/BRAF | |
|---|---|---|---|
| Cell line | Tissue | mutation | Resistance |
| H3255 | Lung | EGFR - L858R | Erlotinib sensitive |
| | | | (40 nM)^{a} |
| HCC827 | Lung | EGFR - Exon 19 deletion | Erlotinib sensitive |
| | | | (4 nM)^{a} |
| H1975 | Lung | EGFR- L858R/T790M | Erlotinib resistant |
| | | | (4.3 μM)^{a} |
| HCT116 | CRC | KRAS - G13D | Cetuximab |
| | | | resistant^{b} |
| SW48 | CRC | Wild type KRAS/BRAF | Sensitive to |
| | | | cetuximab^{b} |
| SW48G12V | CRC | Isogeneic Hetrozygous | Resistant to |
| | | KRAS mutant | cetuximab^{b} |
| HT29 | CRC | EGFR - Wild type, high | Cetuximab |
| | | expression BRAF - 600E | resistant^{b} |
| RKO | CRC | EGFR- Wild type, low | Cetuximab |
| | | expression BRAF - 600E | resistant^{c} |

D1 Shows Synergy with Cetuximab, U0126 and Chloroquine in NSCLC Cell Lines

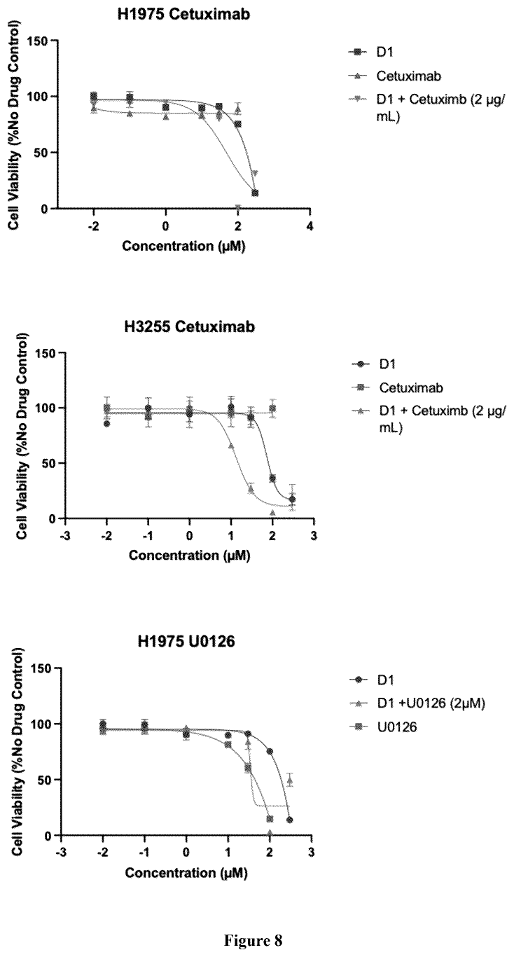

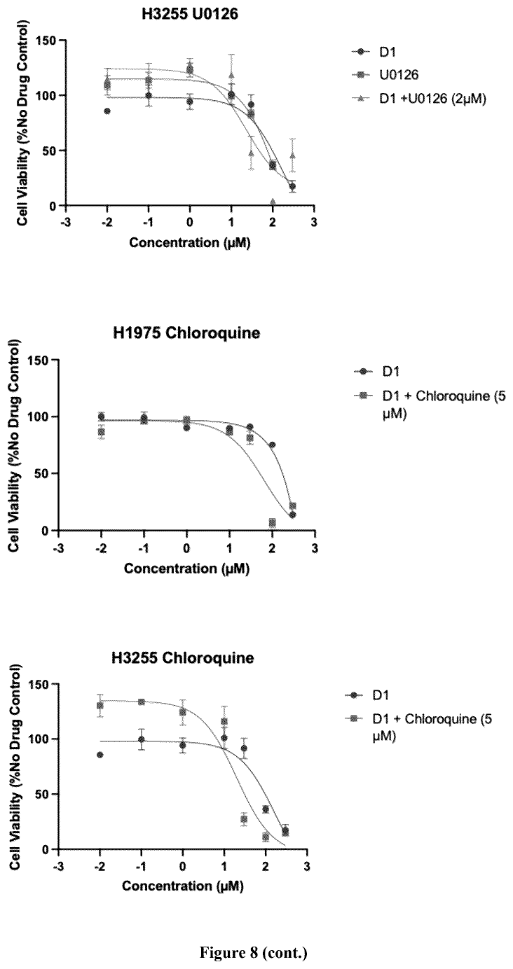

Using D1 as a lead compound, initial experiments were conducted in NSCLC cell lines H3255 (erlotinib sensitive, L858R) and H1975 (erlotinib resistant, L858R/T790M) to determine possible synergy with EGFR inhibitor cetuximab, MEK inhibitor U0126, and clathyrin mediated endocytosis inhibitor chloroquine. D1 exhibited synergy with all three, with potent synergy with cetuximab, in cell lines generally resistant to cetuximab treatment (FIG. 8) (Mukohara et al. 2010, Hu et al. 2016).

D1 Shows Synergy with Cetuximab in Both Sensitive and Resistant CRC Cell Lines

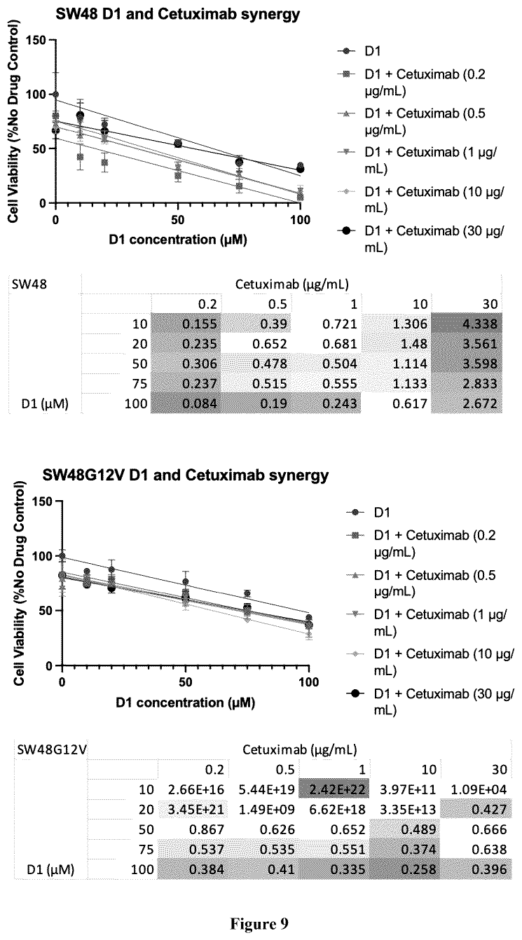

As Cetuximab is clinically used in the treatment of CRC, its synergy was investigated in a pair of isogenic cell lines, SW48 (cetuximab sensitive) and SW48G12V (cetuximab resistant). Cell lines were treated with D1 (0.001-100 μM) in combination with cetuximab (0.02-30 μg/mL) for 72 hours, with significant synergy between the two compounds in both cell lines (FIG. 9).

### DISCUSSION

EGFR's key role in the progression of multiple cancers has led to the development of two main classes of inhibitors—monoclonal antibodies and TKIs. However, mutation-driven resistance often occurs to these therapies, thus limiting their clinical efficacy². There has been recent interest in the development of inhibitors that hijack the internalisation pathway of EGFR, shunting EGFR towards the lysosome for degradation through a non-CME mechanism, rather than recycled back to the membrane via CME. For example, a proof of concept for this was shown with the antibody cetuximab attached to a glycopeptide linker, known as a lysosome targeting chimera (LYTAC)¹². A derivative of the naturally occurring 23-hydroxybetulinic acid known as DPBA also shows EGFR degradation¹³. Here, we have provided evidence that LYTAPs, a new class of small molecule EGFR inhibitor, can result in >90% EGFR degradation.

FLM-C2 was designed from the structure of hGIIA^{14,15}, which has recently been observed as an activating ligand for EGFR^{16,17}, suggesting a likely mechanism for hGIIA's previously observed activation of ERK signalling, as well as cPLA₂-α activation and subsequent eicosanoid production. FLM-C2 is known to inhibit hGIIA-driven eicosanoid production in prostate cancer cells (Mann et al. in review) and the present Example demonstrates the potential mechanism of this function. FLM-c2 blocks hGIIA interaction with EGFR (FIG. 1B), presumably because FLM-c2 is derived from the key region of hGIIA that binds EGFR (FIG. 1a). Further FLM-c2 inhibits EGF binding of EGFR, suggesting the mechanism of action is through the extracellular domain (FIG. 2C).

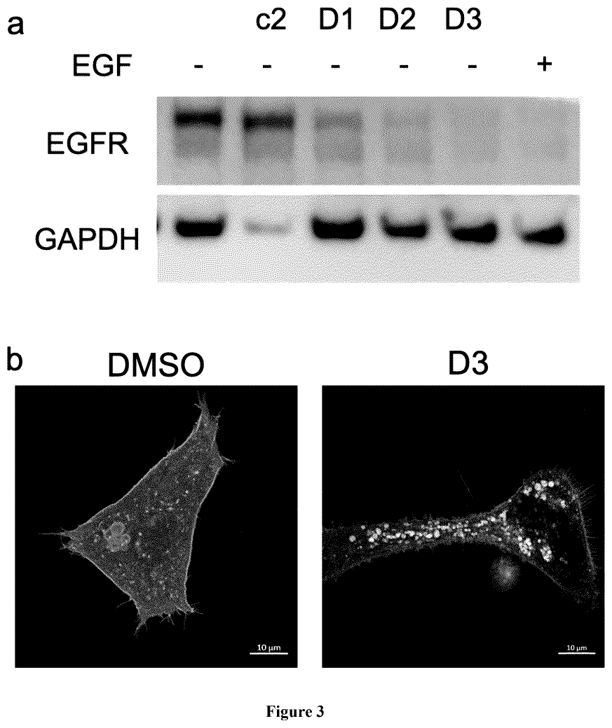

While FLM-c2 inhibits ligand binding to EGFR, it is not considered a LYTAP as it does not initiate lysosomal degradation. The hydrophobic group on the carboxy-side of Arg ((L-(2Nal)) in FLM-c2) does not dock into the extracellular cavity of EGFR's ligand binding domain (FIG. 1C). The key difference between FLM-c2 and its derivatives D1, D2 and D3 is that the derivatives specifically bind the cavity of the EGFR extracellular domain (seq: 23-HFLSLQRM-30, 41-LETT-44, L17) due to their structures (D-(1-Nal) or D-Phe). Molecular modelling indicates this increases the affinity of these compound in comparison to the FLM-c2 analogue (cyclo(Phe-Leu-Ser-Phe-Arg)) (FIG. 1D, FIG. 2B), and it is hypothesised that this slows the ‘off rate’ of the interaction, thus directing EGFR sequestration towards the non-CME pathway and ultimately, lysosomal degradation. The mechanisms via which EGFR is sorted into either CME or non-CME is still not fully understood, but it appears to be dependent on both the binding affinity¹⁸ and concentration of the ligand¹⁹. Since the D1-D3 derivatives initiate EGFR transport to the lysosome (FIG. 3B) resulting in degradation (FIG. 3a), and without being bound by any theory, it is postulated they serve primarily as LYTAPs. A subsequent time course assay indicates that D3 has the most prolonged effect in degrading EGFR, with nearly 98% degraded after 48 hours (FIG. 4B).

The effectiveness of LYTAPs were investigated here in prostate cancer cells, as EGFR is critical to the growth of androgen insensitive prostate cancer as it represents an escape mechanism from inhibition of androgen signalling²⁰. LYTAPs target the extracellular cavity of EGFR, which is a highly conserved region of EGFR, suggesting that it may have broad application in the treatment of other EGFR-driven cancers such as NSCLC, CRC, head and neck cancer, metastatic squamous carcinoma, and glioblastoma.

### REFERENCES

1. Herbst, R. S., 2004. Review of epidermal growth factor receptor biology. International Journal of Radiation Oncology* Biology* Physics, 59(2), pp. S21-S26.
2. Wheeler, D. L., Dunn, E. F. and Harari, P. M., 2010. Understanding resistance to EGFR inhibitors-impact on future treatment strategies. Nature reviews Clinical oncology, 7(9), pp. 493-507.
3. Yarden, Y. and Sliwkowski, M. X., 2001. Untangling the ErbB signalling network. Nature reviews Molecular cell biology, 2(2), pp. 127-137.
4. Karapetis, C. S., Khambata-Ford, S., Jonker, D. J., O'Callaghan, C. J., Tu, D., Tebbutt, N. C., Simes, R. J., Chalchal, H., Shapiro, J. D., Robitaille, S. and Price, T. J., 2008. K-ras mutations and benefit from cetuximab in advanced colorectal cancer. New England Journal of Medicine, 359(17), pp. 1757-1765.
5. De Roock, W., Claes, B., Bernasconi, D., De Schutter, J., Biesmans, B., Fountzilas, G., Kalogeras, K. T., Kotoula, V., Papamichael, D., Laurent-Puig, P. and Penault-Llorca, F., 2010. Effects of KRAS, BRAF, NRAS, and PIK3CA mutations on the efficacy of cetuximab plus chemotherapy in chemotherapy-refractory metastatic colorectal cancer: a retrospective consortium analysis. The lancet oncology, 11(8), pp. 753-762.
6. Montagut, C., Dalmases, A., Bellosillo, B., Crespo, M., Pairet, S., Iglesias, M., Salido, M., Gallen, M., Marsters, S., Tsai, S. P. and Minoche, A., 2012. Identification of a mutation in the extracellular domain of the Epidermal Growth Factor Receptor conferring cetuximab resistance in colorectal cancer. Nature medicine, 18(2), pp. 221-223.
7. Nair, S., Trummell, H. Q., Rajbhandari, R., Thudi, N. K., Nozell, S. E., Warram, J. M., Willey, C. D., Yang, E. S., Placzek, W. J., Bonner, J. A. and Bredel, M., 2020. Novel EGFR ectodomain mutations associated with ligand-independent activation and cetuximab resistance in head and neck cancer. PLoS One, 15(2), p. e0229077.
8. Lin, Y., Wang, X. and Jin, H., 2014. EGFR-TKI resistance in NSCLC patients: mechanisms and strategies. American journal of cancer research, 4(5), p. 411.
9. Sigismund, S., Argenzio, E., Tosoni, D., Cavallaro, E., Polo, S. and Di Fiore, P. P., 2008.

Clathrin-mediated internalization is essential for sustained EGFR signaling but dispensable for degradation. Developmental cell, 15(2), pp. 209-219.
10. Tomas, A., Futter, C. E. and Eden, E. R., 2014. EGF receptor trafficking: consequences for signaling and cancer. Trends in cell biology, 24(1), pp. 26-34.
11. Jones, S., King, P. J., Antonescu, C. N., Sugiyama, M. G., Bhamra, A., Surinova, S., Angelopoulos, N., Kragh, M., Pedersen, M. W., Hartley, J. A. and Futter, C. E., 2020. Targeting of EGFR by a combination of antibodies mediates unconventional EGFR trafficking and degradation. Scientific Reports, 10(1), p. 663.
12. Qu, X., Liu, H., Song, X., Sun, N., Zhong, H., Qiu, X., Yang, X. and Jiang, B., 2021. Effective degradation of EGFRL858R+ T790M mutant proteins by CRBN-based PROTACs through both proteosome and autophagy/lysosome degradation systems. European Journal of Medicinal Chemistry, 218, p. 113328.
13. Yao, N., Wang, C. R., Liu, M. Q., Li, Y. J., Chen, W. M., Li, Z. Q., Qi, Q., Lu, J. J., Fan, C. L., Chen, M. F. and Qi, M., 2020. Discovery of a novel EGFR ligand DPBA that degrades EGFR and suppresses EGFR-positive NSCLC growth. Signal Transduction and Targeted Therapy, 5(1), p. 214.
14. Tseng, A, Inglis, A S, Scott, K F 1996, ‘Native peptide inhibition. Specific inhibition of type II phospholipases A2 by synthetic peptides derived from the primary sequence’, Journal of biological chemistry, vol. 271, no. 39, pp. 23992-23998.
15. Church, WB, Inglis, A S, Tseng, A, Duell, R, Lei, PW, Bryant, KJ, Scott, K F 2001, ‘A novel approach to the design of inhibitors of human secreted phospholipase A(2) based on native peptide inhibition’, Journal of biological chemistry, vol. 276, no. 35, pp. 33156-33164.
16. Dong, Z., Meller, J., Succop, P., Wang, J., Wikenheiser-Brokamp, K., Starnes, S. and Lu, S., 2014. Secretory phospholipase A2-IIa upregulates HER/HER2-elicited signaling in lung cancer cells. International Journal of Oncology, 45(3), pp. 978-984.
17. Lu, S. and Dong, Z., 2017. Overexpression of secretory phospholipase A2-IIa supports cancer stem cell phenotype via HER/ERBB-elicited signaling in lung and prostate cancer cells. International journal of oncology, 50(6), pp. 2113-2122.
18. Zhou, Y. and Sakurai, H., 2022. New trend in ligand-induced EGFR trafficking: A dual-mode clathrin-mediated endocytosis model. Journal of Proteomics, p. 104503.
19. Sigismund, S., Argenzio, E., Tosoni, D., Cavallaro, E., Polo, S. and Di Fiore, P. P., 2008. Clathrin-mediated internalization is essential for sustained EGFR signaling but dispensable for degradation. Developmental cell, 15(2), pp. 209-219.
20. Traish, A. M. and Morgentaler, A., 2009. Epidermal growth factor receptor expression escapes androgen regulation in prostate cancer: a potential molecular switch for tumour growth. British journal of cancer, 101(12), pp. 1949-1956.

1. A cyclic peptide comprising the sequence of the formula:
Xaa¹-Xaa²-Xaa³-Xaa⁴-Xaa⁵
in which:
Xaa¹ is F or 1NapA;
Xaa² is L or I;
Xaa³ is S or T;
Xaa⁴ is F or 2NapA; and
Xaa⁵ is R or K.

2. (canceled)

3. (canceled)

4. The cyclic peptide of claim 1, comprising the sequence of: 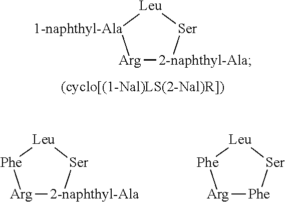
or a pharmaceutically acceptable salt, solvate or prodrug thereof.

5. The cyclic peptide of a claim 4, comprising the chemical structure of: 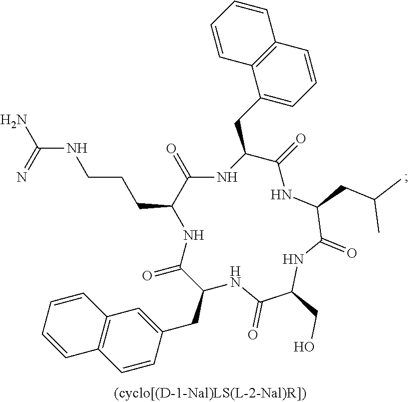 [SMILES: CC(C)C[C@@H]1NC(=O)[C@H](Cc2cccc3ccccc23)NC(=O)[C@H](CCCNC(=N)N)NC(=O)[C@H](Cc2ccc3ccccc3c2)NC(=O)[C@H](CO)NC1=O]
or a pharmaceutically acceptable salt, solvate or prodrug thereof.

6. The cyclic peptide of claim 4, wherein the cyclic peptide is in the form of an acetate salt.

7. A pharmaceutical composition, comprising the cyclic peptide of claim 4 and a pharmaceutically acceptable carrier, diluent or excipient.

8. A method of treating, preventing or ameliorating an EGFR-related disease, disorder or condition in a subject, said method including the step of administering a therapeutically effective amount of a cyclic peptide comprising the sequence of the formula:
Xaa¹-Xaa²-Xaa³-Xaa⁴-Xaa⁵
in which:
Xaa¹ is F or 1NapA;
Xaa² is L or I;
Xaa³ is S or T;
Xaa⁴ is F or 2NapA; and
Xaa⁵ is R or K;
or a pharmaceutically acceptable salt, solvate or prodrug thereof, claim 4 to the subject to thereby treat, prevent or ameliorate the EGFR-related disease, disorder or condition.

9. The method of claim 8, wherein the EGFR-related disease, disorder or condition is an EGFR-related cancer.

10. The method of claim 9, wherein the EGFR-related cancer is prostate cancer, non-small cell lung cancer, pancreatic cancer, ovarian cancer, gastrointestinal cancer, rectal cancer, kidney cancer, liver cancer, gallbladder cancer, head and neck cancer, transitional cell carcinoma, squamous cell carcinoma, melanoma, glioblastoma, gliosarcoma, colorectal cancer, breast cancer, oesophageal cancer, bladder cancer, hepatocellular carcinoma, renal cell carcinoma, brain and central nervous system cancer, neuroendocrine cancer, lymphoma, multiple myeloma, or chronic lymphocytic leukaemia.

11. A method of inhibiting EGFR in a cell, said method including the step of contacting the cell with an effective amount of the cyclic peptide of claim 1 to thereby inhibit EGFR in the cell.

12. A method of promoting internalisation and/or lysosomal degradation of EGFR in a cell, said method including the step of contacting the cell with an effective amount of a cyclic peptide comprising the sequence of the formula:
Xaa¹-Xaa²-Xaa³-Xaa⁴-Xaa⁵
in which:
Xaa¹ is F or 1NapA;
Xaa² is L or I;
Xaa³ is S or T;
Xaa⁴ is F or 2NapA; and
Xaa⁵ is R or K;
or a pharmaceutically acceptable salt, solvate or prodrug thereof, to thereby promote internalisation and/or lysosomal degradation of EGFR in the cell.

13. The method of claim 12, wherein the cell is a cancer cell.

14. A method of reducing the viability and/or growth of a cancer cell, said method including the step of contacting the cancer cell with an effective amount of the cyclic peptide of claim 1 to thereby reduce the viability and/or growth of the cancer cell.

15. The method of claim 13, wherein the cancer cell is a prostate cancer cell, a non-small cell lung cancer cell, a pancreatic cancer cell, an ovarian cancer cell, a gastrointestinal cancer cell, a rectal cancer cell, a kidney cancer cell, a liver cancer cell, a gallbladder cancer cell, a head and neck cancer cell, a transitional cell carcinoma cell, a squamous cell carcinoma cell, a melanoma cell, a glioblastoma cell, a gliosarcoma cell, a colorectal cancer cell, a breast cancer cell, an oesophageal cancer cell, a bladder cancer cell, a hepatocellular carcinoma cell, a renal cell carcinoma cell, a brain and central nervous system cancer cell, a neuroendocrine cancer cell, a lymphoma cell, a multiple myeloma cell, or a chronic lymphocytic leukaemia cell.

16. The method of claim 13, wherein the cancer cell is an EGFR-related cancer cell.

17-20. (canceled)

21. A method of identifying, designing or producing an agent capable of interacting with or binding to a ligand binding domain of EGFR, said method including the steps of:
(a) contacting a cell expressing EGFR with a candidate agent, wherein the candidate agent is a derivative, variant or analogue of: (i) cyclo-((2-Nal)-Leu-Ser-(2-Nal)-Arg); or (ii) a cyclic peptide of the formula:
Xaa¹-Xaa²-Xaa³-Xaa⁴-Xaa⁵
in which:
Xaa¹ is F or 1NapA;
Xaa² is L or I;
Xaa³ is S or T:
Xaa⁴ is F or 2NapA; and
Xaa⁵ is R or K;
or a pharmaceutically acceptable salt, solvate or prodrug thereof; and
(b) determining whether the candidate agent inhibits the binding of an EGFR ligand to EGFR of the cell.

22-29. (canceled)

30. An agent capable of interacting with or binding to a ligand binding domain of EGFR, obtained by the method of claim 21.

31. A method of manufacturing the cyclic peptide of claim 1, wherein said method comprises solid phase synthesis.

32. The method of claim 9, wherein the EGFR-related cancer is prostate cancer, non-small cell lung cancer or colorectal cancer.

33. The method of claim 9, wherein the EGFR-related cancer is at least partly resistant or refractory to an anti-EGFR antibody.

34. The method of claim 33, wherein the EGFR-related cancer is at least partly resistant or refractory to cetuximab.

35. The method of claim 9, wherein the EGFR-related cancer is at least partly resistant or refractory to a small molecule EGFR inhibitor.

36. The method of claim 35, wherein the EGFR-related cancer is at least partly resistant or refractory to erlotinib.

37. The method of claim 9, further including the step of administering a therapeutically effective amount of a further anti-cancer agent to the subject.

38. The method of claim 37, wherein the further anti-cancer agent is a further EGFR inhibitor or a MEK inhibitor.

39. The method of claim 38, wherein the further EGFR inhibitor is an anti-EGFR antibody.

40. The method of claim 39, wherein the further EGFR inhibitor is cetuximab.

41. The method of claim 38, wherein the further EGFR inhibitor is a small molecule EGFR inhibitor.

42. The method of claim 41, wherein the further EGFR inhibitor is erlotinib.
